(19) United States
(12) Patent Application Publication
(10) Pub. No.: US 2002/0159917 A1
(43) Pub. Date: Oct. 31, 2002

(54) System and method for cleaning, high level disinfection, or sterilization of medical or dental instruments or devices

(76) Inventors: Sally Kay Swart, Rosemount, MN (US); Shaun Patrick Kennedy, North Oaks, MN (US)

Correspondence Address: MERCHANT & GOULD PC, P.O. BOX 2903, MINNEAPOLIS, MN, 55402-0903, US

(21) Appl. No.: 09/844,965

(22) Filed: Apr. 27, 2001

(51) Int. Cl. C23F 11/18; A61L 2/00; B08B 3/00; B06B 1/00; B32B 5/02; B32B 27/04; B32B 27/12; A61L 9/00

(52) U.S. Cl. 422/20; 422/18; 422/128; 422/297; 422/300; 134/61; 134/85; 134/95.2; 134/95.3

(57) ABSTRACT

The present invention includes a method and system for cleaning, disinfecting, or sterilizing objects, such as medical or dental instruments or devices. The system of the invention includes an energy source, such as a sonicator, adapted and configured to impact the object with energy, such as ultrasonic energy; a liquid transporter adapted and configured to circulate around, through, and/or into the object a wash composition, an antimicrobial composition, a rinse composition, or a plurality of these compositions; and a dryer adapted and configured to dry the object, preferably, in the presence of a sterilant. The method of the invention includes contacting the object with a wash composition and energy, such as ultrasonic energy; treating the object with an antimicrobial composition; rinsing the object with a rinse composition; and drying the object, preferably in the presence of a sterilant.

### FIELD OF THE INVENTION

The present invention includes a method and system for cleaning, disinfecting, or sterilizing objects, such as medical or dental instruments or devices. The system of the invention includes an energy source, such as a sonicator, adapted and configured to impact the object with energy, such as ultrasonic energy; a liquid transporter adapted and configured to circulate around, through, and/or into the object a wash composition, an antimicrobial composition, a rinse composition, or a plurality of these compositions; and a dryer adapted and configured to dry the object, preferably, in the presence of a sterilant. The method of the invention includes contacting the object with a wash composition and energy, such as ultrasonic energy; treating the object with an antimicrobial composition; rinsing the object with a rinse composition; and drying the object, preferably in the presence of a sterilant.

### BACKGROUND OF THE INVENTION

Numerous apparatus wash and disinfect objects such as medical or dental instruments or devices. Apparatus that wash and disinfect generally require that any stubborn soil be manually removed by presoaking followed by manual scrubbing. A typical apparatus that washes and disinfects applies a cleaning composition and a disinfectant to an object. Some washing and disinfecting apparatus employ a sonication bath. After washing and disinfecting, the object is subjected to sterilization, typically by steam in an autoclave or with ethylene oxide gas.

Other apparatus wash and terminally disinfect or sterilize objects such as medical or dental instruments or devices. After the wash and rinse cycles, such an apparatus exposes the object to high temperature and pressure steam. However, stubborn soils must still be removed by a presoak with manual scrubbing.

There remains a need for an apparatus or system that dislodges stubborn soils from objects such as medical or dental instruments or devices without the need for manual scrubbing, and that sterilizes these objects without steam.

### SUMMARY OF THE INVENTION

The present invention includes a method and system for cleaning, disinfecting, or sterilizing objects, such as medical or dental instruments or devices. The system of the invention includes an energy source, such as a sonicator, adapted and configured to impact the object with energy, such as ultrasonic energy; a liquid transporter adapted and configured to circulate around, through, and/or into the object a wash composition, an antimicrobial composition, a rinse composition, or a plurality of these compositions; and a dryer adapted and configured to dry the object, preferably, in the presence of a sterilant.

The sonicator can be adapted and configured to provide ultrasonic energy throughout fluid within the system, to provide ultrasonic energy from one or more points within the system, to provide ultrasonic energy from one or more points outside the system. A sonicator that provides ultrasonic energy throughout fluid within the system preferably includes a tank or bath type sonicator. A sonicator that provides ultrasonic energy from one or more points can include a sonicator probe constructed for immersion in a liquid or an irrigated probe. Preferably, the system also includes a holder that can position the object near the probe or in a position suitable for cleaning with the probe. The system can include more than one sonicator, for example, to advantageously provide both ultrasonic energy throughout a fluid and from a probe.

The liquid transporter can include one or more independent liquid transporter systems or subsystems for circulating one or more fluids at one or more stations of the system. The liquid transporter can be constructed to immerse the object in a liquid composition or to spray the object with the composition. One liquid transporter can circulate several liquid compositions and include the valves and controllers required for changing compositions. Alternatively, a composition can have a dedicated liquid transporter. Preferably, the liquid transporter is adapted and configured to circulate wash, rinse, and antimicrobial compositions in a defined sequence.

The dryer is adapted and configured to dry the object, preferably in the presence of a sterilant. The dryer can include or work in conjunction with the apparatus that dispenses the sterilant. Typically, the dryer circulates heated gas, such as heater air, around the object. Sterilant can be added before, during, or after drying, for example, by an injector that releases gaseous sterilant from a pressurized vessel. The dryer can include venting adapted and configured to remove the heated gas and the sterilant from the surroundings of the object and, optionally, from the system. Alternatively, sterilant injector and vent can be distinct from and operate independently of the dryer. With such a sterilant injector and vent, exposing the object to sterilant can occur before, during, or after drying. Advantageously, the gaseous sterilant does not wet the object after drying and the object emerges from the system dry.

The system can include one or more stations adapted and configured to circulate a composition around the object and/or to dry the object. Preferably, at least one of the stations includes a housing that can enclose the object. The stations can occupy any of several locations with respect to one another. The stations can share or have distinct subassemblies, such as plumbing (e.g. sprayers, pumps, etc.). For example, stations can occupy the same space or chamber, but employ different apparatus for circulating compositions. In another configuration, stations can occupy overlapping spaces. For example, stations can occupy overlapping locations in a housing of a pass-through or tunnel unit. Stations can also occupy distinct spaces.

Typically the first station is constructed for impacting the object with ultrasonic energy. The first station and the liquid transporter are adapted and configured to circulate at the first station and around the object a wash composition, an antimicrobial composition, a rinse composition, or to circulate a plurality of these compositions. In a one station embodiment, the first station and the dryer can be adapted and configured to dry the object at the first station.

The second station is adapted and configured to circulate the antimicrobial composition, the rinse composition, or a plurality of these compositions. In a two station embodiment, the second station and the dryer can be adapted and configured to dry the object at the second station.

The third station is adapted and configured to circulate the antimicrobial composition, the rinse composition, or a plurality of these compositions. In a three station embodiment, the third station and the dryer can be adapted and configured to dry the object at the third station.

The fourth station is adapted and configured to circulate the antimicrobial composition, the rinse composition, or a plurality of these compositions. In a four station embodiment, the fourth station and the dryer can be adapted and configured to dry the object at the fourth station.

The fifth station is adapted and configured to circulate the rinse composition, and typically, is adapted and configured to dry the object and/or to expose the object to gaseous sterilant.

The sixth station is adapted and configured to dry the object and/or to expose the object to gaseous sterilant.

The method of the invention includes contacting the object with a wash composition and energy, such as ultrasonic energy; treating the object with an antimicrobial composition; rinsing the object with a rinse composition; and drying the object, preferably in the presence of a sterilant.

The wash composition can include an antimicrobial agent, e.g., a quaternary ammonium antimicrobial agent, and a mild detergent, a source of alkalinity, e.g. carbonate, or a combination thereof. Contacting the object with ultrasonic energy can include providing ultrasonic energy from a point source adjacent the object, applying ultrasonic energy throughout the wash composition, or both. Contacting with wash composition typically includes circulating a wash composition around the object.

Treating with antimicrobial composition can include treating with a peroxycarboxylic acid composition. Antimicrobial treating typically includes treating the object for a sufficient time to achieve high level disinfection, or for a sufficient time to achieve sterilization.

Rinsing typically employs a rinse composition including an alcohol drying agent, a lubricant, a rinse agent, or a combination thereof. Rinsing can include a first rinsing after contacting and before treating, a second rinsing after treating and before drying, or a combination thereof. The first rinsing can include rinsing with water, a rinse agent, or a combination thereof. The second rinsing can include rinsing with an alcohol drying agent, a lubricant, a rinse agent, or a combination thereof.

Drying can include exposing the object to a gaseous sterilant and/or circulating a heated gas around the object. Alternatively, exposing the object to a gaseous sterilant can occur independently of, and before or after, drying.

### BRIEF DESCRIPTION OF THE DRAWINGS

FIG. 1 is a block diagram of a one station embodiment of the system of the invention with separate or overlapping stations.

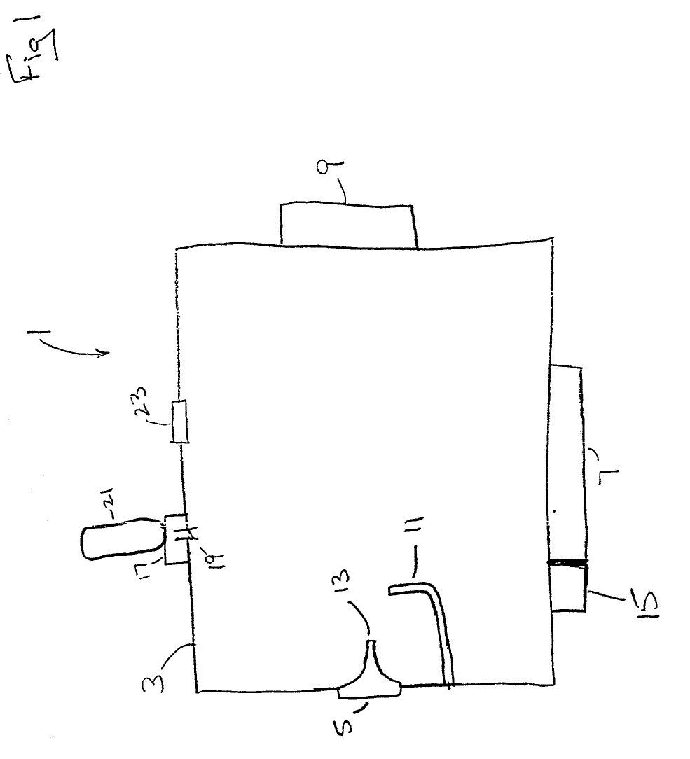

FIG. 2 is a block diagram of a two station embodiment of the system of the invention with separate or overlapping stations.

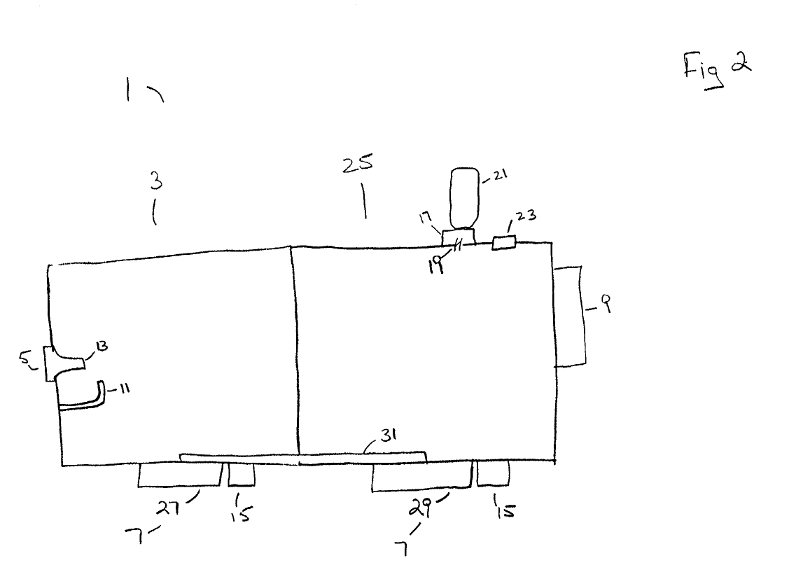

FIG. 3 is a block diagram of a three station embodiment of the system of the invention with separate or overlapping stations.

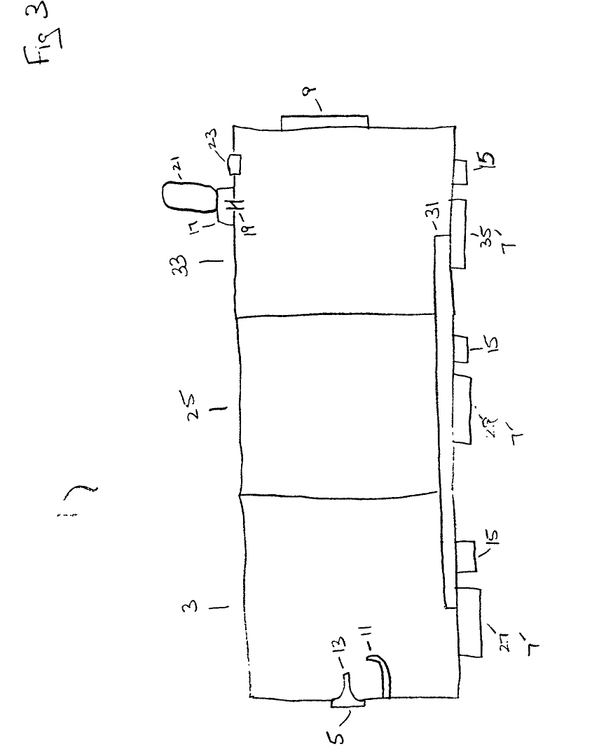

FIG. 4 is a block diagram of a four station embodiment of the system of the invention with separate or overlapping stations.

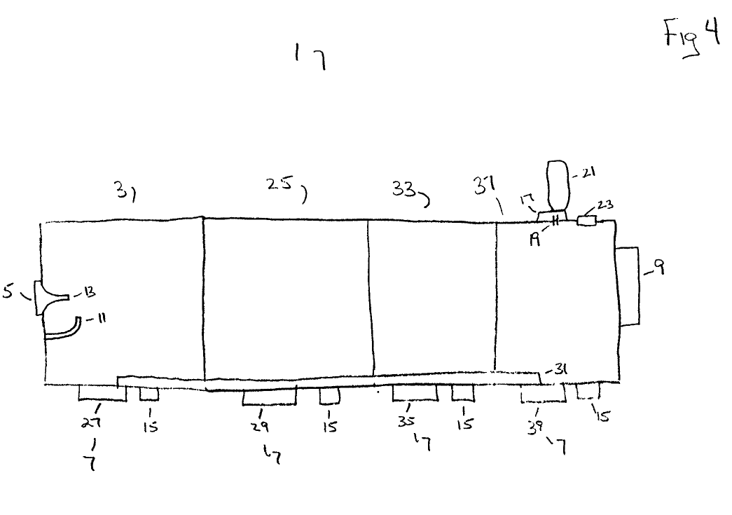

FIG. 5 is a block diagram of a five station embodiment of the system of the invention with separate or overlapping stations.

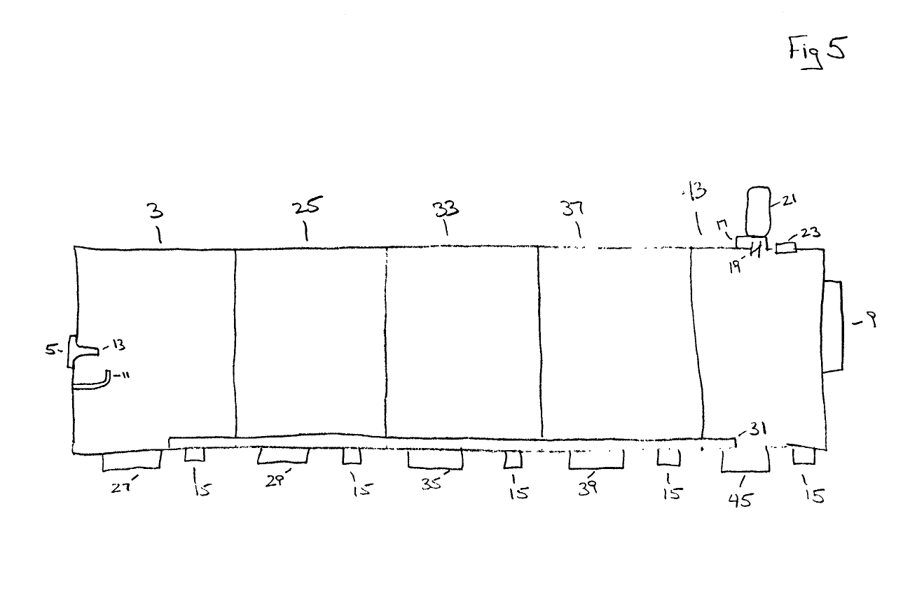

FIG. 6 is a block diagram of a six station embodiment of the system of the invention with separate or overlapping stations.

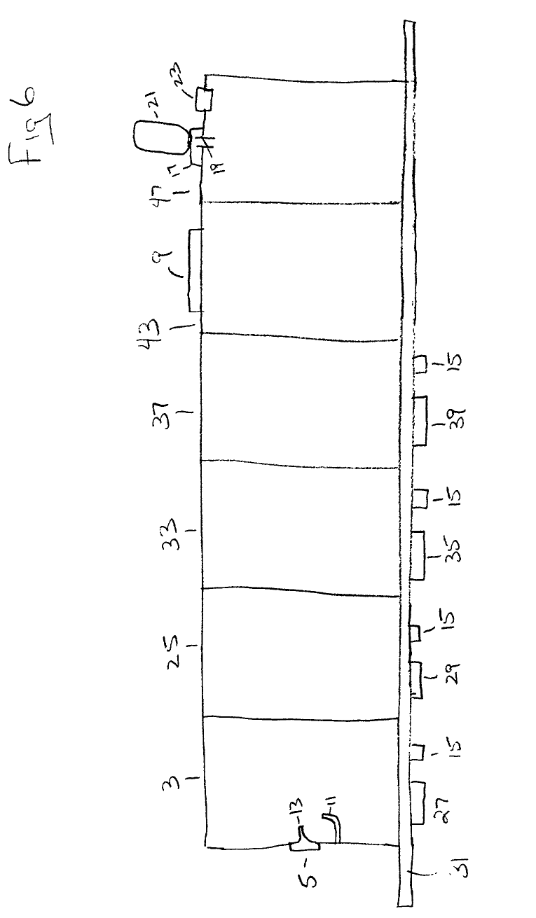

FIG. 7 is a block diagram of a five station embodiment of the system of the invention with separate or overlapping stations and including a plurality of emitters.

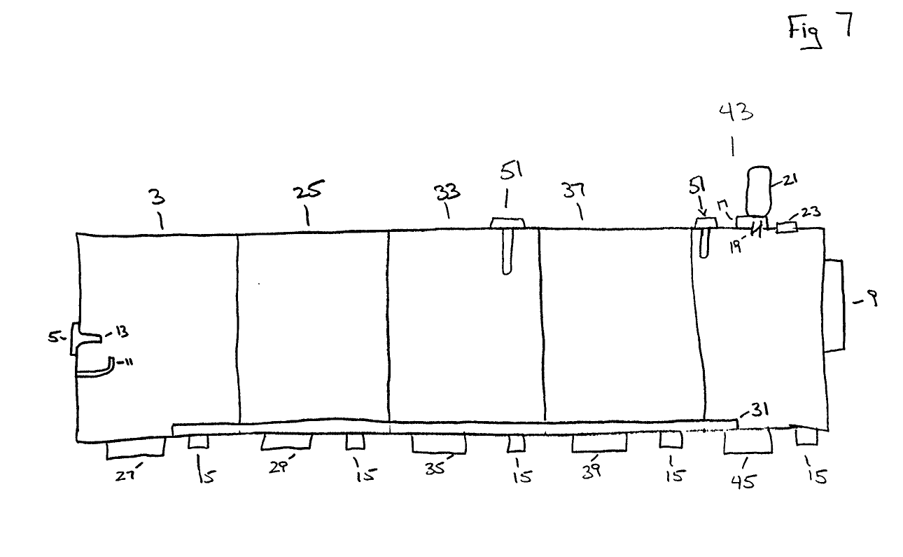

FIG. 8 is a block diagram of a five station embodiment of the system of the invention with the stations in the same or overlapping spaces.

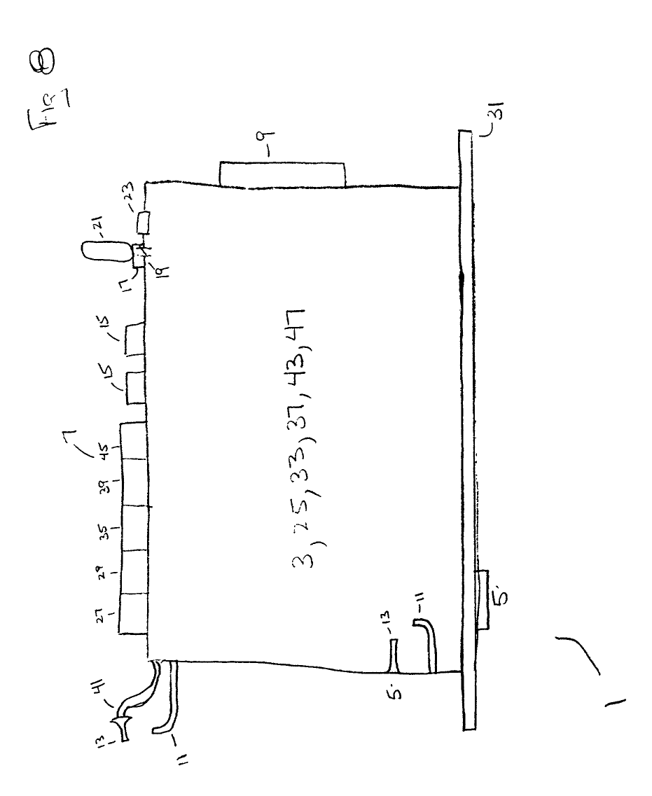

### DETAILED DESCRIPTION OF THE INVENTION

Definitions

As used herein, the term “around” refers to on all of various sides, including the exterior of and within any accessible portions of an object, or surrounding an object. For example, for an object having cavities or lumens that are or can open to the exterior of the object, around includes into those cavities or lumens. Thus, around includes in and around, within, through, and throughout an object. Around includes around any accessible solid portion of the object. As used herein, circulating around an object includes circulating around the exterior of the object and into or through any accessible portions of the object, such as any cavities or lumens. For example, circulating fluid around an object includes circulating fluid through any lumen or into any accessible cavity. Circulating around and object with one or more lumens or cavities can include employing apparatus, e.g., as part of the liquid transporter, to direct fluid into the cavity or lumen.

As used herein, the term “instrument” refers to the various medical or dental instruments or devices that can benefit from cleaning, disinfection, and/or sterilization.

As used herein, the phrases “medical instrument”, “dental instrument”, “medical device”, “dental device”, “medical equipment”, or “dental equipment” refer to instruments, devices, tools, appliances, apparatus, and equipment used in medicine or dentistry. Such instruments, devices, and equipment can be cold sterilized, soaked or washed and then heat sterilized, or otherwise benefit from cleaning, disinfecting, or sterilizing according to the present invention. These various instruments, devices and equipment include, but are not limited to: diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, endoscopes (e.g., noninvasive flexible and rigid fiber optic endoscopes), endotracheal tubes, anesthesia breathing circuits, cytoscopes, arthoscopes and related equipment, and the like, or combinations thereof.

As used herein, elevated temperature refers to temperatures above room temperature and commonly employed for washing or presoaking wares or instruments. Washing can typically be conducted at elevated temperatures of about 20 to about 80° C., preferably about 30 to about 60° C., preferably about 60 to about 80° C. Rinsing can typically be conducted at elevated temperatures of about 40 to about 80° C. High temperature rinsing can typically be conducted at elevated temperatures of about 80 to about 95° C.

As used herein, the terms “decontamination” or “decontaminate” refer to use of physical or chemical processes to remove, inactivate, or destroy pathogenic organisms on an object or its surface and to render the object safe for handling, use of disposal.

As used herein, the terms “disinfection” or “disinfect” refer to destruction of pathogenic and other microorganisms by thermal or chemical processes destroying most pathogens, but not necessarily all microbial forms, such as bacterial spores.

As used herein, the terms “sterilization” or “sterilize” refer to processes used to render an object free of all forms of viable microorganisms.

As used herein, the term “microorganisms” refers to any noncellular or unicellular (including colonial) organism. Microorganisms include all prokaryotes. Microorganisms include bacteria (including cyanobacteria), lichens, microfungi, protozoa, virinos, viroids, viruses, and some algae. As used herein, the term “microbe” is synonymous with microorganism.

Washing and Disinfecting System

The present invention includes a system and method for washing, disinfecting, and/or sterilizing an object, such as a medical device or instrument. The system can include an emitter that applies energy to the object, such as a sonicator that applies ultrasonic energy to the object; one or more liquid transporter systems that circulate around the object a wash composition, a rinse composition, and/or an antimicrobial composition; and/or a dryer constructed to dry the object, preferably in the presence of a sterilant. In general terms, the system includes apparatus for converting a soiled object, e.g., a soiled medical or dental instrument or device, to a cleaned, disinfected, and/or sterilized object.

The system can be constructed to expose the object to ultrasonic energy, or another type of energy, before and/or during a wash cycle. For example, the system can employ an ultrasonic probe, such as an irrigated ultrasonic probe, positioned near the object to expose the object to ultrasonic energy either before running the wash cycle and/or during the wash cycle. The sonicator can include a moveable, external probe, such as an irrigated probe, for manual sonic cleaning of dirty objects.

The system can be constructed to circulate wash composition around the object with concurrent application of ultrasonic energy. For example, the system can include a sprayer that sprays the object with wash composition while a sonicator applies ultrasonic energy. Alternatively, by way of further example, the system can include an immersion tank, a circulating pump that circulates wash composition within the immersion tank, a sonicator that applies ultrasonic energy to the circulating wash composition, and a conveyor that transports the object into or through the tank. The system can include a water heater or other apparatus to increase the temperature of the wash composition to an elevated temperature.

The wash composition employed in the wash cycle typically includes a surfactant or other cleaner, but can also include an antimicrobial agent, such as a quaternary ammonium antimicrobial agent. Optionally, the wash composition includes an enzyme, such as a detersive enzyme.

After washing, optionally, the system can employ the same or similar spraying or immersion apparatus to circulate around the object a first rinse composition. The first rinse composition removes wash composition and loose soil from the object, and can include a rinse agent.

Next, employing the same or similar spray or immersion apparatus, the system can circulate around the object an antimicrobial composition, such as a peroxycarboxylic acid composition. A preferred peroxycarboxylic acid composition includes builders and/or corrosion inhibitors and has a pH of about 6. The antimicrobial composition can be employed at a concentration, temperature, and for a time sufficient to achieve antimicrobial effect, high level disinfection, or sterilization. The system can include a water heater or other apparatus to maintain the temperature of the antimicrobial composition at or slightly above room temperature.

The system can include an emitter adapted and configured to impact the object with energy either during or after circulating antimicrobial composition. Such an emitter can impact the object with, for example, light or microwave energy, preferably in the presence of the antimicrobial composition. Although not limiting to the present invention, it is believed that impacting with such energy has antimicrobial effects and can also generate radicals and other antimicrobial moieties from the antimicrobial composition, which increases the effectiveness of circulating the antimicrobial composition.

The system can then employ the same or similar spraying or immersion apparatus to circulate around the object a second rinse composition, preferably a rinse composition including alcohol drying agent and/or an oil based emulsion or “milk bath” lubricant for medical instruments. Lubricating oil based emulsions or milk baths are known to those of skill in the art.

The system also can include a dryer constructed to dry the object, preferably, in the presence of a sterilant. For example, the dryer can include a blower that circulates ambient or heated air around the object. The circulated air can include a gaseous sterilant, such as chlorine dioxide or ethylene oxide. The gaseous sterilant can be added to the surroundings of the object by an injector that releases the gas from a pressure vessel. This injector can be a part of the dryer, or it can be an independent apparatus. The injector can be adapted and configured to release gaseous sterilant into the surroundings of the object before, after, or during drying. The system can include an emitter adapted and configured to impact the object with energy before, during, or after operation of the dryer.

The present invention relates to a system and method for washing and disinfecting an object. System (1) includes a sonicator (5), a liquid transporter (7), and a dryer (9). Dryer (9) is adapted and configured to dry the object, preferably in the presence of a sterilant. Liquid transporter (7) is adapted and configured to circulate fluids around the object. Liquid transporter (7) can transport fluids such as a wash composition, antimicrobial composition, a rinse composition, a plurality of these compositions, or preferably, all of these compositions. Sonicator (5) is adapted and configured to impact the object with ultrasonic energy. Typically the antimicrobial and/or rinse compositions are circulated by liquid transporter (7) in the form of a sprayer (not shown).

Typically, system (1) includes one or more stations that can house the object during the several washing, rinsing, disinfecting, and drying cycles. In an embodiment, the system includes a first station (3), sonicator (5), liquid transporter (7), and dryer (9). Dryer (9) is adapted and configured to dry the object, preferably in the presence of a sterilant. Liquid transporter (7) is adapted and configured to circulate fluids at the first station and around the object. Liquid transporter (7) can transport fluids such as a wash composition, antimicrobial composition, a rinse composition, or a plurality of these compositions. Sonicator (5) is adapted and configured to impact the object in the first station (3) with ultrasonic energy.

Sonicator (5) can be constructed in any of several configurations. Sonicator (5) can be adapted and configured to provide ultrasonic energy throughout fluid within system (1), e.g. at first station (3). For example, configurations of sonicators (5) suitable for sonication tanks or baths are known to those of skill in the art. Alternatively, sonicator (5) can be adapted and configured to provide ultrasonic energy from one or more points within system (1), e.g. first station (3). For example, configurations of ultrasonic probes that emit ultrasonic energy, for example from a location within a liquid, are known to those of skill in the art. In a preferred embodiment, the system also includes a holder (11) for an instrument, which can position the instrument for receiving ultrasonic energy. In this embodiment, sonicator (5) can include ultrasonic probe (13) within system (1), e.g., first station (3), and adjacent a position taken by the instrument in holder (11). Sonicator (5) typically dislodges soil from an object or instrument in the system, and the soil can be removed from the object or instrument by the liquid transporter (7).

System (1) and/or sonicator (5) can include second sonicator (41) positioned on the exterior of system (1) and/or external to first station (3). Second sonicator (41) preferably includes a probe adapted and configured to be positioned near the object. For example, in an embodiment, the second sonicator (41) probe can be positioned near holder (11) positioned on the exterior of system (1) and used to manually sonicate the object. Preferably, second sonicator (41) probe (13) is moveable and can be positioned near a particular soiled portion of the object. Preferably, second sonicator (41) probe (13) is an irrigated probe. Alternatively, system (1) can include a liquid bath for immersion of the object and second sonicator (41) probe (13).

Preferably, sonicator (5) or second sonicator (41) is adapted and configured to provide ultrasonic energy throughout a liquid bath at about 8 to about 300 kHz, preferably about 15 to about 60 kHz. Preferably, sonicator (5) or second sonicator (41) is adapted and configured to provide ultrasonic energy from a point source, such as a probe (13) or an irrigated probe, at about 20 to about 30 kHz.

Liquid transporter (7) can be constructed in any of several configurations. Liquid transporter (7) circulates one or more fluids, typically liquids, within the system (1), e.g., at first station (3), and around the object. Circulating includes any way in which a fluid, particularly a liquid, can be moved through system (1) and over, in, and/or around the object. Circulating includes moving fluid through or into any cavities or lumens in or on the object. For example, if the object is immersed in a liquid, circulating can include stirring, pumping, or agitating liquid at or within the station. Typically, such stirring, pumping, or agitating moves the liquid relative to the object to carry dislodged soil away from the object. Typically, such stirring, pumping, or agitating moves the liquid into or through any cavities or lumens that might be present in or on the object. Thus, liquid transporter (7) can include stirrers, pumps, or paddles that circulate liquid within the station.

Circulating fluid around an object can also include moving, with a pump or other mechanism, liquid through the lumen of an object, such as anesthesia tubing or a catheter. For objects with one or more lumen, liquid transporter (7) can be adapted and configured to couple to the object to direct fluid through the lumen. For example, anesthesia tubing or a catheter can be oriented in rack that positions the lumen into the flow of the composition or that couples the lumen to a jet circulating the composition.

Circulating around an object can also include spraying liquid at system (1) and over or around the object. Circulating around an object by spraying can include spraying directed at or into any cavity or lumen that might be present on the device. Typically, spraying includes employing a sprayer having a nozzle, jet, arm, or wand (not shown). The sprayer can include fixed or moving spray jets or arms, e.g. it can be a rotary sprayer. The sprayed liquid can be drained from system (1) or recovered by liquid transporter (7) and sprayed again. Liquid transporter (7) can include a pump that recovers liquid from the bottom of system (1) and/or forces it through a spray nozzle, jet, or wand (not shown).

Liquid transporter (7) can also add a fluid to system (1), e.g., at first station (3), and/or remove fluid from system (1), e.g., at first station (3). Liquid transporter (7) can sequentially circulate a wash composition, an antimicrobial composition, and/or a rinse composition. Sequentially circulating refers to circulating one composition after another. Typically, sequential circulation includes adding a first composition to system (1), circulating the first composition within system (1), removing the first composition from system (1), adding a second composition to system (1), circulating the second composition within system (1), removing the second composition from system (1), and so on. Typically sequentially circulating includes circulating in the order listed.

In an embodiment, system (1), preferably first station (3), sonicator (5), and liquid transporter (7) are adapted and configured to immerse the object in the wash composition and apply ultrasonic energy to the immersed object. Sonicator (5) can be adapted and configured to provide ultrasonic energy from a point within system (1), to apply ultrasonic energy throughout the wash composition, or both. In configurations in which the object is immersed in a liquid in system (1), particularly first station (3), system (1) or station (3) can take any of a variety of configurations suitable for containing a liquid. For example, system (1) or station (3) can include a tub, a tank, or a bath. In configurations in which the object is immersed in a liquid in system (1), particularly first station (3), liquid transporter (7) can include one or more pumps, drains, and/or valves configured to add liquid to system (1), circulate liquid within system (1) and around the object (including into or through any cavities or lumens), and to drain or remove liquid from system (1). A tub, tank or bath employed in system (1) can include a sump that receives the wash, rinse, or antimicrobial composition, and that also includes a heating coil for heating one or more of these compositions. In configurations in which system (1), particularly first station (3), immerses the object in a liquid, system (1) can include a conveyor, lift, or other apparatus adapted and configured to lower the object or a rack containing the object into the tub, tank, or bath containing the composition.

System (1), e.g., first station (3), can also circulate around the object antimicrobial composition and/or rinse the object. Rinsing can include rinsing with a first rinse composition after washing and rinsing with a second rinse composition before drying. The rinse composition can include either or both of the first and second rinse compositions. For example, system (1), e.g., first station (3), and liquid transporter (7) can be adapted and configured to immerse the object one or more times in one or more batches of the antimicrobial composition, the rinse composition, or a plurality of these compositions. Alternatively, liquid transporter (7) can include a sprayer (not shown). The sprayer can be adapted and configured to apply to and circulate around the object the antimicrobial composition, the rinse composition, or a plurality of these compositions. Spraying can include sequential spraying with different or the same batches of the same composition. Spraying can occur in a spray chamber portion of system (1), e.g., first station (3).

The system of the invention can also include one or more dispensers (15) adapted and configured to add to a fluid employed in the system a wash concentrate, an antimicrobial concentrate, a rinse concentrate, or to add a plurality of these concentrates. Numerous types of dispensers (15) for liquid and/or solid wash, antimicrobial, or rinse compositions, and/or for apparatus for washing and disinfecting medical or dental instruments or devices, are known to those of skill of the art and can be employed in the present system. Dispenser (15) for liquid compositions can dilute a liquid concentrate composition to form a second concentrate, which can be diluted again to form a use composition. Dispenser (15) for liquid compositions can dilute a liquid concentrate composition directly to form a use composition. Dispenser (15) for solid compositions can dissolve or suspend a solid composition to form a liquid concentrate, which can be diluted to form a use composition. Dispenser (15) for solid compositions can dissolve or suspend a solid composition directly to form a use composition.

Dryer (9) can be constructed in any of several configurations. For example, dryer (9) can be adapted and configured to dry the object while the object is in system (1), e.g., at first station (3). Dryer (9) can dry the object using drying forces such as radiant heat, a circulated, dry gas (e.g. heated air), or steam. In an embodiment, dryer (9) comprises a blower (not shown) adapted and configured to circulate a heated gas within system (1), e.g., at first station (3). Dryer (9) can vent used heated gas to the surroundings of system (1), or can remove moisture from the used heated gas and reuse this dried gas.

The system of the invention can include an apparatus (49) adapted and configured to add the sterilant to system (1), e.g., at first station (3), preferably as a component of dryer (9). The apparatus (49) can add sterilant before, during, or after drying. Preferably, a sterilant employed after drying does make the object wet. Exposing to sterilant before or after drying can occur in the same location or station as drying, or in a different location or station from drying.

An apparatus (49) adapted and configured to add the sterilant can include an injector (17) adapted and configured to add sterilant to system (1), e.g., at first station (3). The sterilant can be either a gas or a liquid, preferably a gas. For a gaseous sterilant, injector (17) can include a pressure release valve (19) adapted and configured to release the gaseous sterilant from a pressurized vessel (21) into system (1), e.g., at first station (3). Typically, a system employing a gaseous sterilant will also include a vent (23) adapted and configured to evacuate sterilant gas from system (1), e.g., at first station (3). Vent (23) can expel evacuated gas to the surroundings of system (1), or send the evacuated gas through a filter for reuse or retention in system (1). Preferably, system (1) employs a gas tight chamber, or a chamber under negative pressure, for the gaseous sterilant, and employs filters or other ventilation systems that prevent exposure of an operator or bystander to the gaseous sterilant.

System (1) can also include one or more temperature control systems that heat and/or cool and regulate the temperature of the wash, rinse, and/or antimicrobial compositions, or of the air or other fluid employed for drying. For example, the temperature control systems can include in line or tank heaters, such as coil heaters, for the wash, rinse, and/or antimicrobial compositions, or for water employed to form these compositions. Similarly, electric elements can be employed to dry and/or heat the air, sterilant, or other fluid employed for drying and disinfecting. The temperature control systems can include thermostats or other sensors that detect the temperature of a composition or other fluid and that signal a processor or other controller to regulate the temperature of the composition or other fluid.

System (1) can also include mechanical, analog, or digital control apparatus to start and stop in the appropriate sequence the various components of the system, such as liquid transporters (e.g., sprayers or pumps), conveyors, if any, sonicators, and dryers. Such mechanical, analog, or digital control apparatus for cleaning systems is well-known to those of skill in the art. The control apparatus can include systems for determining which of the several sonic, wash, rinse, and antimicrobial cycles to run for a particular object or rack of objects. Such a control apparatus can include a bar code reader that reads a bar code on a rack or object, which details the cycles to run for that rack or object. The control apparatus can also include safety mechanisms linked to one or more doors of system (1) that prevent system (1) from running unless the doors are sealed or closed, or if unauthorized objects are in system (1).

System (1) can also include a load apparatus or unload apparatus. Load apparatus supports and moves objects or racks containing objects into system (1). Unload apparatus receives racks exiting system (1). The unload apparatus can be coupled to the load apparatus to return racks to the load apparatus, for example, by conveyor. System (1) can also include a conveyor (31), which can move the object or a rack containing the object through system (1). In a manual embodiment, an operator can place the object or a rack containing the object into system (1), and remove it from system (1).

System (1) can also include one or more emitters (51). Emitter (51) can impact the object with energy, such as microwave or light energy, inside or outside the system. Emitter (51) can be adapted and configured to impact the object with energy before circulation of wash composition. For example, emitter (51) can impact the object with energy before it is placed in to a station or chamber to reduce the burden of microbes or soil. By way of further example, emitter (51) can provide energy throughout the chamber or station before wash composition is circulated.

Emitter (51) can be adapted and configured to impact the object with energy during circulation of the wash composition. For example, emitter (51) can impact the object with light, preferably ultraviolet light, energy from throughout the wash composition. By way of further example, emitter (51) can impact the object with light, preferably ultraviolet light, energy from a point in the chamber or station concurrently with circulation of the wash composition. Emitter can be adapted and configured to impact the object with energy during or after circulation of the antimicrobial composition. Such an emitter (51) can impact the object with, for example, light or microwave energy, preferably in the presence of the antimicrobial composition. For example, emitter (51) can impact the object with light, preferably ultraviolet light, energy from throughout the antimicrobial composition. By way of further example, emitter (51) can impact the object with light, preferably ultraviolet light, energy or microwave energy from a point in the chamber or station after with circulation of the antimicrobial composition.

Emitter (51) can be adapted and configured to impact the object with energy before, after, or during operation of the dryer. For example, after circulation of (second) rinse composition, emitter (51) can impact the object with energy. For example, during operation of the dryer, emitter (51) can impact the object with energy. For example, after operation of the dryer, emitter (51) can impact the object with energy. Such impacting with energy before, during, or after operation of the dryer can substitute for or, preferably, supplement, exposing the object to gaseous sterilant.

In an embodiment, system (1) includes sonicator (5), liquid transporter (7), and dryer (9). Sonicator (5) is adapted and configured to impact the object with ultrasonic energy. Liquid transporter (7) is adapted and configured to circulate around the object a wash composition, an antimicrobial composition, and a rinse composition. Dryer (9) is adapted and configured to dry the object, preferably in the presence of a sterilant.

In this embodiment, sonicator (5) can take on a variety of configurations. For example, sonicator (5) can be adapted and configured to provide ultrasonic energy throughout fluid in system (1). Alternatively, sonicator (5) can be adapted and configured to provide ultrasonic energy from a point within system (1). System (1) can also include holder (11), which can be adapted and configured to support the object, with sonicator (5) including an ultrasonic probe adjacent a position taken by the object in holder (11). Preferably, in such a configuration, sonicator (5) includes an irrigated probe. Sonicator (5) can, optionally, also include a second sonicator (41) located on the exterior of system (1). Second sonicator (41) can include a probe outside the system (1). Preferably, in such a configuration, second sonicator (41) includes an irrigated probe. Preferably, in such a configuration, the system also includes holder (11) adapted and configured to support the object adjacent the probe of second sonicator (41).

In an embodiment, system (1) includes sonicator (5), liquid transporter (7,) and dryer (9). Sonicator (5) is adapted and configured to impact the object with ultrasonic energy. Liquid transporter (7) is adapted and configured to circulate around the object a wash composition, an antimicrobial composition, and a rinse composition. Dryer (9) is adapted and configured to dry the object, preferably in the presence of a sterilant.

In this embodiment, sonicator (5) can be adapted and configured to provide ultrasonic energy throughout fluid in the system. Sonicator (5) and liquid transporter (7) can be adapted and configured to immerse the object in the wash composition and apply ultrasonic energy to the immersed object. System (1) can also include a holder (11). Holder (11) is preferably adapted and configured to support the object. When the system includes a holder (11), sonicator (5) preferably includes an ultrasonic probe adjacent a position taken by the object in the holder (11). The ultrasonic probe can be an irrigated probe. System (1) can also include a second sonicator (41). Second sonicator (41) can be adapted and configured to provide ultrasonic energy from a probe on the exterior of the system (1). This probe can also be an irrigated probe. A system (1) with a second sonicator (41) typically also includes a holder (11) adapted and configured to support the object adjacent the second sonicator probe.

In an embodiment, system (1) includes a station, e.g., first station (3), holder (11), sonicator (5), liquid transporter (7), dispenser (15), dryer (9), injector (17), and vent (23). Holder (11) extends within the station, e.g., first station (3), and is adapted and configured to support the object. Sonicator (5) is adapted and configured to impact the object in the station with ultrasonic energy. Liquid transporter (7) is adapted and configured to circulate at the station and around the object a wash composition and a rinse composition. Liquid transporter (7) includes a sprayer adapted and configured to apply the rinse composition to the object. Dispenser (15) is adapted and configured to add a wash concentrate and a rinse concentrate. Dryer (9) is adapted and configured to dry the object in the station, preferably in the presence of the sterilant. Injector (17) includes a pressure release valve adapted and configured to release a gaseous sterilant from a pressurized vessel into the station. The vent (23) is adapted and configured to evacuate sterilant gas from the station. Sonicator (5) can be adapted and configured to provide ultrasonic energy throughout fluid in the first station. Alternatively, sonicator (5) can be adapted and configured to provide ultrasonic energy from a point within the station.

In an embodiment, system (1) includes a station, e.g., first station (3), holder (11), sonicator (5), liquid transporter (7), and dryer (9). Holder (11) extends within the station, e.g., first station (3), and is adapted and configured to support the object. Sonicator (5) is adapted and configured to provide ultrasonic energy from a point within the station. Liquid transporter (7) is adapted and configured to circulate at the station and around the object a wash composition and a rinse composition. Dryer (9) is adapted and configured to dry the object in the station, preferably in the presence of the sterilant.

In an embodiment, system (1) includes a station, e.g., first station (3), sonicator (5), liquid transporter (7), and dryer (9). Sonicator (5) is adapted and configured to provide ultrasonic energy throughout fluid in the station. Liquid transporter (7) is adapted and configured to circulate at the station and around the object a wash composition and a rinse composition. Dryer (9) is adapted and configured to dry the object in the station, preferably in the presence of the sterilant.

System (1) can include two, three, four, five, six, or more stations. Embodiments with additional stations are described hereinbelow.

Two Station Embodiments

System (1) can include first station (3) and a second station (25). In a two station configuration, typically, first station (3) includes sonicator (5) and is adapted and configured to circulate at first station (3) and around the object the wash composition, the antimicrobial composition, the rinse composition, or a plurality of these compositions. In such a configuration, second station (25) is typically adapted and configured to circulate at second station (25) and around the object the antimicrobial composition, the rinse composition, or a plurality of these compositions. Sonicator (5), liquid transporter (7), holder (11) and other system (1) components can adopt configurations described hereinabove for system (1) or one station embodiments. Although first station (3) and second station (25) can each circulate one or more of the same compositions, this generally does not occur. Preferably, first station (3), sonicator (5), and liquid transporter (7) are adapted and configured to immerse the object in the wash composition and apply ultrasonic energy to the immersed object within first station (3), as described hereinabove.

Second station (25) and liquid transporter (7) can be adapted and configured to immerse the object in the antimicrobial composition, the rinse composition, or a plurality of these compositions. Alternatively, liquid transporter (7) can include a sprayer (not shown). The sprayer can be adapted and configured to apply to the object at second station (25) the antimicrobial composition, the rinse composition, or a plurality of these compositions. Spraying can occur in a spray chamber portion of second station (25), which can be the entirety of this station. Liquid transporter (7) can include a pump and a valve adapted and configured to circulate at second station (25) and around the object the antimicrobial composition, the rinse composition, or both compositions; to add to second station (25) the antimicrobial composition, the rinse composition, or both compositions; and to remove from second station (25) the antimicrobial composition, the rinse composition, or both compositions. A two station embodiment can also include a dispenser (15) adapted and configured to add an antimicrobial concentrate and/or a rinse concentrate to fluid employed at second station (25).

In embodiments in which second station (25) circulates one or more fluids, liquid transporter (7) can be adapted and configured to circulate fluids at second station (25). Such a liquid transporter (7) can include a single system that circulates fluids in both first station (3) and second station (25) or separate subsystems for each of the stations. For example, liquid transporter (7) can include a first transporter subsystem (27), which circulates fluids at first station (3), and a second transporter subsystem (29), which circulates fluids at second station (25). Alternatively, the system of the invention can include distinct liquid transporters (7) for each system (1). Features of liquid transporter (7) are described hereinabove.

In an embodiment, second station (25) can be adapted and configured to circulate the rinse composition. In this embodiment, first station (3) can preferably be adapted and configured to circulate the wash composition and/or the antimicrobial composition. Liquid transporter (7) circulates rinse composition at second station (25) and over the object. Preferably, in this embodiment, first station (3) can be adapted and configured to circulate around the object the wash composition, the antimicrobial composition, and first rinse composition. First station (3) preferably can be adapted and configured to sequentially circulate, in this order, the wash composition, the first rinse composition, and the antimicrobial composition. Preferably, the second station (25) can be adapted and configured to circulate around the object second rinse composition. Second station (25) and liquid transporter (7) can be adapted and configured to immerse the object in the rinse composition. Alternatively, liquid transporter (7) can include a sprayer. The sprayer can be adapted and configured to apply the rinse composition to the object. Spraying can occur in a spray chamber portion of second station (25), which can be the entirety of this system (1). Liquid transporter (7) can include a pump and a valve adapted and configured to circulate rinse composition at second station (25), to add rinse composition to second station (25), and to remove rinse composition from second station (25). This embodiment can also include a dispenser (15) adapted and configured to add a rinse concentrate to fluid employed in the second station.

In an embodiment, second station (25) can be adapted and configured to circulate the antimicrobial composition and the rinse composition. In this embodiment, first station (3) can preferably be adapted and configured to circulate the wash composition. Liquid transporter (7) circulates antimicrobial composition and rinse composition at second station (25) and over the object. In this embodiment, first station (3) can be adapted and configured to circulate around the object the wash composition and first rinse composition. Second station (25) can be adapted and configured to circulate around the object the antimicrobial composition and second rinse composition. Preferably, the embodiment employs sequential circulation. For example, liquid transporter (7) can be adapted and configured to sequentially circulate at the second station the antimicrobial composition followed by the rinse composition. By way of further example, example, second station (25) and liquid transporter (7) can be adapted and configured to immerse the object in the antimicrobial composition and the rinse composition, preferably sequentially. Alternatively, liquid transporter (7) can include a sprayer (not shown). The sprayer can be adapted and configured to apply to the object the antimicrobial composition and the rinse composition, preferably sequentially. Spraying can occur in a spray chamber portion of second station (25), which can be the entirety of this station. Liquid transporter (7) can include a pump and a valve adapted and configured to circulate at the second station the antimicrobial composition, the rinse composition, or both compositions; to add to the second station the antimicrobial composition, the rinse composition, or both compositions; and to remove from the second station the antimicrobial composition, the rinse composition, or both compositions. This embodiment can also include a dispenser (15) adapted and configured to add an antimicrobial concentrate and/or a rinse concentrate to fluid employed in the second station.

In a two station embodiment, drying generally occurs in the second station (25). Thus, second station (25) can include dryer (9). Various features of dryer (9) are described hereinabove. Second station (25) can also include the apparatus (49) that adds sterilant to and vents sterilant from system (1). Such apparatus (49) is also described hereinabove. In an embodiment, second station (25) includes dryer (9) and does not include a liquid transporter (7). In this embodiment, first station (3) includes liquid transporter (7), sonicator (5), and other apparatus that washes, accomplishes antimicrobial treatment, and rinses (as described hereinabove).

In a two station embodiment, the two stations can occupy any of several locations with respect to one another. The two stations can share or have distinct subassemblies, such as plumbing (e.g. sprayers, pumps, etc.). For example, first station (3) and second station (25) can occupy the same space or chamber, but employ different apparatus for circulating compositions. In such an embodiment, system (1) can include a single housing. An operator loads the object(s) into the housing, the object remains stationary in the housing, and first and second stations (3 and 25) circulate compositions around and through the object. Such a single housing system (1) can employ first and second stations (3 and 25) to apply compositions that benefit from different types of application (e.g., immersing and spraying), or that benefit from liquid handling apparatus (e.g. liquid transporter (7) including pumps, piping, and the like) made from different materials or to different specifications. In an embodiment of single housing system (1), first and second stations (3 and 25) can include a (the same) spray chamber, or, for example, a (the same) tub, tank, or bath.

In another configuration, first station (3) and second station (25) can occupy overlapping spaces. For example, first station (3) and second station (25) can occupy overlapping locations in a housing of a pass-through or tunnel unit. As the object translocates through the housing, it first encounters a region where first station (3) circulates a composition around and through the object. Then the object encounters a region where it contacts the compositions circulated by first station (3) and by second station (25). As the object moves further through the housing, it encounters a region where second station (25), but not first station (3), circulates a composition around the object.

By way of further example, first station (3) and second station (25) can occupy distinct spaces. In one embodiment, first station (3) and second station (25) can occupy distinct locations in a housing of a pass-through or tunnel unit. As the object translocates through the housing, it first encounters a location where first station (3) circulates a composition around and through the object. Then, the object moves further through the housing, it encounters a distinct location where second station (25), but not first station (3), circulates a composition around and through the object. With such distinct locations, an object would not encounter a region where it simultaneously contacts both of the compositions circulated by first station (3) and by second station (25). Alternatively, first station (3) and second station (25) can occupy distinct housings. For example, first station (3) can include a tub, tank or bath suitable for immersing the object, and second station (25) can include a spray chamber suitable for spraying the object or a gas-tight chamber suitable for exposing the object to a gaseous sterilant.

A two station embodiment can include an apparatus for moving the object from first station (3) to second station (25). For example, the system can include a conveyor (31) adapted and configured to translocate the object between first and second stations (3 and 25) and/or through system (1). First station (3) and second station (25) can occupy the same or overlapping spaces. For configurations in which first and second stations (3 and 25) occupy the same space, conveyor (31) need not translocate the object from one station to the other. In such a configuration, conveyor (31) can translocate the object in and out of, or through, system (1). For configurations in which first and second stations (3 and 25) occupy overlapping spaces, conveyor (31) can translocate the object from a location preferable for circulating a composition at first station (3) to a position preferably for circulating a composition at second station (25). In a position preferable for circulating a composition at first station (3), a composition circulating at second station (25) can still contact the object, and vice versa. In this configuration, conveyor (31) can translocate the object in and out of, or through, system (1). First station (3) and second station (25) can occupy distinct spaces, and, in such an embodiment, conveyor (31) can translocate the object from one station to the other. In this configuration, conveyor (31) can translocate the object in and out of, or through, system (1).

In a manual embodiment, an operator moves the object between first and second stations (3 and 25) or into and out of, or through, system (1).

A two station embodiment can include one or more emitters (51) as described hereinabove for the system and one station embodiments.

In an embodiment, system (1) includes first station (3) and second station (25), sonicator (5), liquid transporter (7), and dryer (9). Sonicator (5) is adapted and configured to impact the object at first station (3) with ultrasonic energy. Liquid transporter (7) is adapted and configured to circulate at first station (3) and around the object a wash composition, an antimicrobial composition, a rinse composition, or a plurality of these compositions. Dryer (9) is adapted and configured to dry the object at the second station (25), preferably in the presence of the sterilant.

In this embodiment, sonicator (5) can take on a variety of configurations. For example, sonicator (5) can be adapted and configured to provide ultrasonic energy throughout fluid in the first station. Alternatively, sonicator (5) can be adapted and configured to provide ultrasonic energy from a point within the station. System (1) can also include holder (11), which can be adapted and configured to support the object, with sonicator (5) including an ultrasonic probe adjacent a position taken by the object in holder (11). Preferably, in such a configuration, sonicator (5) includes an irrigated probe. Sonicator (5) can, optionally, also include a second sonicator (41) located on the exterior of system (1), e.g. first station (3). Second sonicator (41) can include a probe outside the system (1), e.g., first station (3). Preferably, in such a configuration, second sonicator (41) includes an irrigated probe. Preferably, in such a configuration, the system also includes holder (11) adapted and configured to support the object adjacent the probe of second sonicator (41).

In this embodiment, liquid transporter (7), or its subsystems, can circulate the wash, rinse, and antimicrobial compositions at the two stations (3 and 25) in any of several combinations. For example, as one combination, liquid transporter (7) can be adapted and configured to circulate at first station (3) and around the object the wash composition, and to circulate at second station (25) and around the object the antimicrobial composition and the rinse composition. In this combination, liquid transporter (7) can circulate at first station (3) and around the object the wash composition and a first rinse composition, and circulate at the second station (25) and around the object the antimicrobial composition and a second rinse composition. Alternatively, in this combination, liquid transporter can be adapted and configured to circulate at first station (3) and around the object the wash composition; and to circulate at the second station (25) and around the object a first rinse composition, the antimicrobial composition, and a second rinse composition. In another combination, liquid transporter (7) can be adapted and configured to circulate at first station (3) and around the object the wash composition and the antimicrobial composition, and to circulate at second station (25) and around the object the rinse composition. In this combination, liquid transporter (7) can be adapted and configured to circulate at first station (3) and around the object the wash composition, a first rinse composition, and the antimicrobial composition; and to circulate at second station (25) and around the object a second rinse composition.

Three Station Embodiments

System (1) can include first station (3), second station (25), and a third station (33). In such a configuration, typically, first station (3) includes sonicator (5) and is adapted and configured to circulate at first station (3) and around the object the wash composition, the antimicrobial composition, the rinse composition, or a plurality of these compositions. In such a configuration, second station (25) is typically adapted and configured to circulate at second station (25) and around the object the antimicrobial composition, the rinse composition, or a plurality of these compositions. In such a configuration, third station (33) is typically adapted and configured to circulate at third station (33) and around the object the antimicrobial composition, the rinse composition, or a plurality of these compositions. Although first station (3) and second station (25) can each circulate one or more of the same compositions, this generally does not occur. Although first station (3) and third station (33) can each circulate one or more of the same compositions, this generally does not occur. Although second station (25) and third station (33) can each circulate one or more of the same compositions, this generally does not occur.

Sonicator (5), liquid transporter (7), holder (11) and other system (1) components can adopt configurations described hereinabove for system (1) or one and two station embodiments. Preferably, first station (3), sonicator (5), and liquid transporter (7) are adapted and configured to immerse the object in the wash composition and apply ultrasonic energy to the immersed object at first station (3), as described hereinabove. Preferably, second station (25) is adapted and configured to circulate the antimicrobial composition, the rinse composition, or a plurality of these compositions, as described hereinabove.

Third station (33) and liquid transporter (7) can be adapted and configured to immerse the object in the antimicrobial composition, the rinse composition, or a plurality of these compositions. Alternatively, liquid transporter (7) can include a sprayer (not shown). The sprayer can be adapted and configured to apply to the object at third station (33) the antimicrobial composition, the rinse composition, or a plurality of these compositions. Spraying can occur in a spray chamber portion of third station (33), which can be the entirety of this station. Liquid transporter (7) can include a pump and a valve adapted and configured to circulate at third station (33) the antimicrobial composition, the rinse composition, or both compositions; to add to third station (33) the antimicrobial composition, the rinse composition, or both compositions; and to remove from third station (33) the antimicrobial composition, the rinse composition, or both compositions. A three station embodiment can also include a dispenser (15) adapted and configured to add an antimicrobial concentrate and/or a rinse concentrate to fluid employed at third station (33).

In embodiments in which third station (33) circulates one or more fluids, liquid transporter (7) can be adapted and configured to circulate fluids at third station (33) and around the object. Such a liquid transporter (7) can include a single system that circulates fluids at first station (3), second station (25), and third station (33), or separate subsystems for different stations. For example, liquid transporter (7) can include first transporter subsystem (27), which circulates fluids at first station (3), second transporter subsystem (29), which circulates fluids at second station (25), and/or third transporter subsystem (35), which circulates fluids at third station (33). Alternatively, the system of the invention can include distinct liquid transporters (7) for each station. Features of liquid transporter (7) are described hereinabove.

In an embodiment, third station (33) can be adapted and configured to circulate around the object the rinse composition. In this embodiment, first station (3) and second station (25) can preferably be adapted and configured to circulate around the object the wash composition and/or the antimicrobial composition. Liquid transporter (7) circulates rinse composition at third station (33) and around the object. For example, third station (33) and liquid transporter (7) can be adapted and configured to immerse the object in the rinse composition. Alternatively, liquid transporter (7) can include a sprayer. The sprayer can be adapted and configured to apply the rinse composition to the object. Spraying can occur in a spray chamber portion of third station (33), which can be the entirety of this station. Liquid transporter (7) can include a pump and a valve adapted and configured to circulate rinse composition at third station (33), to add rinse composition to third station (33), and to remove rinse composition from third station (33). This embodiment can also include a dispenser (15) adapted and configured to add a rinse concentrate to fluid employed at third station (33).

In an embodiment, third station (33) can be adapted and configured to circulate around the object the antimicrobial composition and the rinse composition. In this embodiment, first station (3) can preferably be adapted and configured to circulate around the object the wash composition. Second station (25) can be adapted and configured to circulate around the object wash and/or antimicrobial compositions. Liquid transporter (7) circulates antimicrobial composition and rinse composition at third station (33) and around the object. Preferably, the embodiment employs sequential circulation. For example, third station (33) and liquid transporter (7) can be adapted and configured to immerse the object in the antimicrobial composition and the rinse composition, preferably sequentially. Alternatively, liquid transporter (7) can include a sprayer (not shown). The sprayer can be adapted and configured to apply to the object the antimicrobial composition and the rinse composition, preferably sequentially. Spraying can occur in a spray chamber portion of third station (33), which can be the entirety of this station. Liquid transporter (7) can include a pump and a valve adapted and configured to circulate at the third station (33) the antimicrobial composition, the rinse composition, or both compositions; to add to the third station (33) the antimicrobial composition, the rinse composition, or both compositions; and to remove from the third station (33) the antimicrobial composition, the rinse composition, or both compositions. This embodiment can also include a dispenser (15) adapted and configured to add an antimicrobial concentrate and/or a rinse concentrate to fluid employed in the third station.

In a three station embodiment, drying generally occurs at third station (33). Thus, third station (33) can include dryer (9). Various features of dryer (9) are described hereinabove. Third station (33) can also include the apparatus (49) that adds sterilant to and vents sterilant from system (1). Such apparatus (49) is also described hereinabove. In an embodiment, third station (33) includes dryer (9) and does not include a liquid transporter (7). In this embodiment, first station (3) and/or second station (25) include liquid transporter (7), sonicator (5), and other apparatus that washes, accomplishes antimicrobial treatment, and rinses (as described hereinabove).

In a three station embodiment, the three stations can occupy any of several locations with respect to one another. The three stations can share or have distinct subassemblies, such as plumbing (e.g. sprayers, pumps, etc.). For example, first station (3), second station (25), and third station (33) can occupy the same space, but employ different apparatus for circulating compositions. This embodiment can employ a configuration analogous to the configuration described hereinabove for two station embodiments. In another configuration, first station (3), second station (25), and/or third station (33) can occupy overlapping spaces. Typically third station (33) and second station (25) occupy overlapping spaces in a configuration analogous to that described hereinabove for two station embodiments. In yet another configuration, first station (3), second station (25), and third station (33) can occupy distinct spaces. Such configurations are analogous to those described hereinabove for two station embodiments. In embodiments in which one or more of the stations occupy the same space, second and third stations (25 and 33) or first, second, and third stations (3, 25, and 33) can include a (the same) spray chamber, or, for example, a (the same) tub, tank, or bath.

A three station embodiment can include an apparatus for moving the object from first station (3) to second station (25), from second station (25) to third station (33), among any other combination of stations, and/or through system (1). For example, system (1) can include a conveyor (31) adapted and configured to move the object between first, second, and/or third stations (3, 25, and 33), and/or through system (1). The three station embodiments can employ conveyor systems with capabilities and structures analogous to those described hereinabove for two station embodiments. In a manual embodiment, an operator moves the object between first, second, and/or third stations (3, 25, and 33).

A three station embodiment can include one or more emitters (51) as described hereinabove for the system and one station embodiments.

In an embodiment, system (1) includes first station (3), second station (25), third station (33), sonicator (5), liquid transporter (7), and dryer (9). Sonicator (5) is adapted and configured to impact the object at first station (3) with ultrasonic energy. Liquid transporter (7) is adapted and configured to circulate at first station (3) and around the object a wash composition, an antimicrobial composition, a rinse composition, or a plurality of these compositions. Liquid transporter (7) is adapted and configured to circulate at second station (25) and around the object an antimicrobial composition, a rinse composition, or a plurality of these compositions. Dryer (9) is adapted and configured to dry the object at the third station (33), preferably in the presence of the sterilant.

In this embodiment, sonicator (5) can take on the variety of configurations described hereinabove for system (1), one, and/or two station embodiments.

In this embodiment, liquid transporter (7), or its subsystems, can circulate the wash, rinse, and antimicrobial compositions at the three stations (3, 25, and 33) in any of several combinations. With each combination, first station (3) includes sonicator (5). As one combination, liquid transporter (7) can be adapted and configured to circulate at first station (3) and around the object the wash composition, a first rinse composition and the antimicrobial composition; and to circulate at second station (25) and around the object a second rinse composition. In another combination, liquid transporter (7) can be adapted and configured to circulate at first station (3) and around the object the wash composition and a first rinse composition, and to circulate at second station (25) and around the object the antimicrobial composition and a second rinse composition. As another combination, liquid transporter (7) can be adapted and configured to circulate at first station (3) and around the object the wash composition; and to circulate at second station (25) and around the object a first rinse composition, the antimicrobial composition, and a second rinse composition. In yet another combination, liquid transporter (7) can be adapted and configured to circulate at first station (3) and around the object the wash composition and a first rinse composition, to circulate at second station (25) and around the object the antimicrobial composition, and to circulate at third station (33) and around the object a second rinse composition. As yet another combination, liquid transporter (7) can be adapted and configured to circulate at first station (3) and around the object the wash composition, to circulate at second station (25) and around the object a first rinse composition and the antimicrobial composition, and to circulate at third station (33) and around the object a second rinse composition.

Four Station Embodiments

System (1) can include first station (3), second station (25), third station (33), and a fourth station (37). In such a configuration, typically, first station (3) includes sonicator (5) and is adapted and configured to circulate at first station (3) and around the object the wash composition, the antimicrobial composition, the rinse composition, or a plurality of these compositions. In such a configuration, second station (25) is typically adapted and configured to circulate at second station (25) and around the object the antimicrobial composition, the rinse composition, or a plurality of these compositions. In such a configuration, third station (33) is typically adapted and configured to circulate at third station (33) and around the object the antimicrobial composition, the rinse composition, or a plurality of these compositions. In such a configuration, fourth station (37) can be adapted and configured to circulate at fourth station (37) and around the object the antimicrobial composition, the rinse composition, or a plurality of these compositions. Although one or more of the stations (3, 25, 33 and 37) can each circulate one or more of the same compositions, this generally does not occur.

Sonicator (5), liquid transporter (7), holder (11) and other system (1) components can adopt configurations described hereinabove for system (1) or one, two, and three station embodiments. Preferably, first station (3), sonicator (5), and liquid transporter (7) are adapted and configured to immerse the object in the wash composition and apply ultrasonic energy to the immersed object at first station (3), as described hereinabove. Preferably, second station (25) is adapted and configured to circulate around the object the antimicrobial composition, the rinse composition, or a plurality of these compositions, as described hereinabove. Preferably, third station (33) is adapted and configured to circulate around the object the antimicrobial composition, the rinse composition, or a plurality of these compositions, as described hereinabove.

Fourth station (37) and liquid transporter (7) can be adapted and configured to immerse the object in the antimicrobial composition, the rinse composition, or a plurality of these compositions. Alternatively, liquid transporter (7) can include a sprayer (not shown). The sprayer can be adapted and configured to apply to the object at fourth station (37) the antimicrobial composition, the rinse composition, or a plurality of these compositions. Spraying can occur in a spray chamber portion of fourth station (37), which can be the entirety of this station. Liquid transporter (7) can include a pump and a valve adapted and configured to circulate at fourth station (37) and around the object the antimicrobial composition, the rinse composition, or both compositions; to add to fourth station (37) the antimicrobial composition, the rinse composition, or both compositions; and to remove from fourth station (37) the antimicrobial composition, the rinse composition, or both compositions. A four station embodiment can also include a dispenser (15) adapted and configured to add an antimicrobial concentrate and/or a rinse concentrate to fluid employed at fourth station (37).

In embodiments in which fourth station (37) circulates one or more fluids, liquid transporter (7) can be adapted and configured to circulate fluids at fourth station (37). Such a liquid transporter (7) can include a single system that circulates fluids at first station (3), second station (25), third station (33), and fourth station (37), or separate subsystems for different stations. For example, liquid transporter (7) can include first transporter subsystem (27), which circulates fluids at first station (3), second transporter subsystem (29), which circulates fluids at second station (25), third transporter subsystem (35), which circulates fluids at third station (33), and/or fourth transporter subsystem (39), which circulates fluids at fourth station (37). Alternatively, the system of the invention can include distinct liquid transporters (7) for each station. Features of liquid transporter (7) are described hereinabove.

In an embodiment, fourth station (37) can be adapted and configured to circulate around the object the rinse composition. In this embodiment, first station (3) can preferably be adapted and configured to circulate around the object the wash composition. In this embodiment, second station (25) and third station (33) can preferably be adapted and configured to circulate around the object the wash composition and/or the antimicrobial composition. Liquid transporter (7) circulates rinse composition at fourth station (37) and around the object. For example, fourth station (37) and liquid transporter (7) can be adapted and configured to immerse the object in the rinse composition. Alternatively, liquid transporter (7) can include a sprayer. The sprayer can be adapted and configured to apply the rinse composition to the object. Spraying can occur in a spray chamber portion of fourth station (37), which can be the entirety of this station. Liquid transporter (7) can include a pump and a valve adapted and configured to circulate rinse composition at fourth station (37) and around the object, to add rinse composition to fourth station (37), and to remove rinse composition from fourth station (37). This embodiment can also include a dispenser (15) adapted and configured to add a rinse concentrate to fluid employed at fourth station (37).

In an embodiment, fourth station (33) can be adapted and configured to circulate around the object the antimicrobial composition and the rinse composition. In this embodiment, first station (3) can preferably be adapted and configured to circulate around the object the wash composition. Second station (25) can be adapted and configured to circulate around the object wash and/or antimicrobial compositions. Third station (33) can be adapted and configured to circulate around the object antimicrobial and/or rinse compositions. Liquid transporter (7) circulates antimicrobial composition and rinse composition at fourth station (37) and around the object. Preferably, the embodiment employs sequential circulation. For example, fourth station (33) and liquid transporter (7) can be adapted and configured to immerse the object in the antimicrobial composition and the rinse composition, preferably sequentially. Alternatively, liquid transporter (7) can include a sprayer (not shown). The sprayer can be adapted and configured to apply to the object the antimicrobial composition and the rinse composition, preferably sequentially. Spraying can occur in a spray chamber portion of fourth station (37), which can be the entirety of this station. Liquid transporter (7) can include a pump and a valve adapted and configured to circulate at fourth station (37) the antimicrobial composition, the rinse composition, or both compositions; to add to fourth station (37) the antimicrobial composition, the rinse composition, or both compositions; and to remove from fourth station (37) the antimicrobial composition, the rinse composition, or both compositions. This embodiment can also include a dispenser (15) adapted and configured to add an antimicrobial concentrate and/or a rinse concentrate to fluid employed at fourth station (37).

In a four station embodiment, drying generally occurs at fourth station (37). Thus, fourth station (37) can include dryer (9). Various features of dryer (9) are described hereinabove. Fourth station (37) can also include the apparatus (49) that adds sterilant to and vents sterilant from system (1). Such apparatus (49) is also described hereinabove. In an embodiment, fourth station (37) includes dryer (9) and does not include a liquid transporter (7). In this embodiment, first station (3), second station (25), and/or third station (33) include liquid transporter (7), sonicator (5), and other apparatus that washes, accomplishes antimicrobial treatment, and rinses (as described hereinabove).

In a four station embodiment, the four stations can occupy any of several locations with respect to one another. The four stations can share or have distinct subassemblies, such as plumbing (e.g. sprayers, pumps, etc.). For example, first station (3), second station (25), third station (33), and fourth station (37) can occupy the same space, but employ different apparatus for circulating compositions. This embodiment can employ configurations analogous to the configuration described hereinabove for two and three station embodiments. In another configuration, first station (3), second station (25), third station (33), and/or fourth station (37) can occupy overlapping spaces. Typically third station (33) and fourth station (37) occupy overlapping spaces in a configuration analogous to that described hereinabove for two and three station embodiments. In yet another configuration, first station (3), second station (25), third station (33), and/or fourth station (37) can occupy distinct spaces. Such configurations are analogous to those described hereinabove for two and three station embodiments. In embodiments in which one or more of the stations occupy the same space, third and fourth stations (33 and 37); second, third, and fourth stations (25, 33, and 37); or first second, third, and fourth stations (3, 25, 33, and 37), can include a (the same) spray chamber, or, for example, a (the same) tub, tank, or bath.

A four station embodiment can include an apparatus for moving the object from first station (3) to second station (25), from second station (25) to third station (33), from third station (33) to fourth station (37), among any other combination of stations, and/or through system (1). For example, system (1) can include a conveyor (31) adapted and configured to move the object between first, second, third, and/or fourth stations (3, 25, 33, and 37), and/or through system (1). The four station embodiments can employ conveyor systems with capabilities and structures analogous to those described hereinabove for two and three station embodiments. In a manual embodiment, an operator moves the object between first, second, third, and/or fourth stations (3, 25, 33, and 37).

A four station embodiment can include one or more emitters (51) as described hereinabove for the system and one station embodiments.

In an embodiment, system (1) includes first station (3), second station (25), third station (33), fourth station (37), sonicator (5), liquid transporter (7), and dryer (9). Sonicator (5) is adapted and configured to impact the object at first station (3) with ultrasonic energy. Liquid transporter (7) is adapted and configured to circulate at first station (3) and around the object a wash composition, an antimicrobial composition, a rinse composition, or a plurality of these compositions. Liquid transporter (7) is adapted and configured to circulate at second station (25) and around the object an antimicrobial composition, a rinse composition, or a plurality of these compositions. Liquid transporter (7) is adapted and configured to circulate at third station (33) and around the object an antimicrobial composition, a rinse composition, or a plurality of these compositions. Dryer (9) is adapted and configured to dry the object at the fourth station (37), preferably in the presence of the sterilant.

In this embodiment, sonicator (5) can take on the variety of configurations described hereinabove for system (1), one, two, and/or three station embodiments.

In this embodiment, liquid transporter (7), or its subsystems, can circulate the wash, rinse, and antimicrobial compositions at the four stations (3, 25, 33, and 37) in any of several combinations. With each combination, first station (3) includes sonicator (5). As one combination, liquid transporter (7) can be adapted and configured to circulate at first station (3) and around the object the wash composition, to circulate at second station (25) and around the object the antimicrobial composition and a first rinse composition, and to circulate at third station (33) and around the object a second rinse composition. As another combination, liquid transporter (7) can be adapted and configured to circulate at first station (3) and around the object the wash composition and a first rinse composition, to circulate at second station (25) and around the object the antimicrobial composition, and to circulate at third station (33) and around the object a second rinse composition. As yet another combination, liquid transporter (7) can be adapted and configured to circulate at first station (3) and around the object the wash composition, to circulate at second station (25) and around the object a first rinse composition, and to circulate at third station (33) and around the object the antimicrobial composition and a second rinse composition. In still another combination, liquid transporter (7) can be adapted and configured to circulate at first station (3) and around the object the wash composition, to circulate at second station (25) and around the object a first rinse composition, to circulate at third station (33) and around the object the antimicrobial composition, and to circulate at fourth station (37) and around the object a second rinse composition.

Five Station Embodiments

System (1) can include first station (3), second station (25), third station (33), fourth station (37), and a fifth station (43). In such a configuration, typically, first station (3) includes sonicator (5) and is adapted and configured to circulate at first station (3) and around the object the wash composition, the antimicrobial composition, the rinse composition, or a plurality of these compositions. In such a configuration, second station (25) is typically adapted and configured to circulate at second station (25) and around the object the antimicrobial composition, the rinse composition, or a plurality of these compositions. In such a configuration, third station (33) is typically adapted and configured to circulate at third station (33) and around the object the antimicrobial composition, the rinse composition, or a plurality of these compositions. In such a configuration, fourth station (37) can be adapted and configured to circulate at fourth station (37) and around the object the antimicrobial composition, the rinse composition, or a plurality of these compositions. In such a configuration, fifth station (43) can be adapted and configured to circulate at fifth station (43) and around the object the rinse composition, preferably a second rinse composition. Although one or more of the stations (3, 25, 33 and 37) can each circulate one or more of the same compositions, this generally does not occur.

Sonicator (5), liquid transporter (7), holder (11) and other system (1) components can adopt configurations described hereinabove for system (1) or one, two, three, and four station embodiments. Preferably, first station (3), sonicator (5), and liquid transporter (7) are adapted and configured to immerse the object in the wash composition and apply ultrasonic energy to the immersed object at first station (3), as described hereinabove. First station (3) can also be adapted and configured to circulate around the object a first rinse composition. Preferably, second station (25) is adapted and configured to circulate around the object the antimicrobial composition, the rinse composition, or a plurality of these compositions, as described hereinabove. Second station (25) can be adapted and configured to circulate around the object the first rinse composition. Preferably, third station (33) is adapted and configured to circulate around the object the antimicrobial composition, the rinse composition, or a plurality of these compositions, as described hereinabove. Preferably, fourth station (37) is adapted and configured to circulate around the object the antimicrobial composition, the rinse composition, or a plurality of these compositions, as described hereinabove. Fourth station (37) can be adapted and configured to circulate around the object a second rinse composition.

Fifth station (43) and liquid transporter (7) can be adapted and configured to immerse the object in the rinse composition, preferably a second rinse composition. Alternatively, liquid transporter (7) can include a sprayer (not shown). The sprayer can be adapted and configured to apply to the object at fifth station (43) the rinse composition, preferably a second rinse composition. Spraying can occur in a spray chamber portion of fifth station (43), which can be the entirety of this station. Liquid transporter (7) can include a pump and a valve adapted and configured to circulate at fifth station (43) and around the object the rinse composition, preferably a second rinse composition; to add to fifth station (43) the rinse composition, preferably a second rinse composition; an d to remove from fifth station (43) the rinse composition, preferably a second rinse composition. A five station embodiment can also include a dispenser (15) adapted and configured to add a rinse concentrate to fluid employed at fifth station (43).

In embodiments in which fifth station (43) circulates one or more fluids, liquid transporter (7) can be adapted and configured to circulate fluids at fifth station (43). Such a liquid transporter (7) can include a single system that circulates fluids at first station (3), second station (25), third station (33), fourth station (37), and fifth station (43), or separate subsystems for different stations. For example, liquid transporter (7) can include first transporter subsystem (27), which circulates at first station (3), second transporter subsystem (29), which circulates fluids at second station (25), third transporter subsystem (35), which circulates fluids at third station (33), fourth transporter subsystem (39), which circulates fluids at fourth station (37), and/or fifth transporter subsystem (45), which circulates fluids at fifth station (43). Alternatively, the system of the invention can include distinct liquid transporters (7) for each station. Features of liquid transporter (7) are described hereinabove.

In an embodiment, fifth station (43) can be adapted and configured to circulate the rinse composition, preferably the second rinse composition. In this embodiment, first station (3) can preferably be adapted and configured to circulate the wash composition. In this embodiment, second station (25), third station (33), and fourth station (37) can preferably be adapted and configured to circulate the wash composition, the first rinse composition, and/or the antimicrobial composition. Liquid transporter (7) circulates the second rinse composition at fifth station (37) and over the object. For example, fifth station (43) and liquid transporter (7) can be adapted and configured to immerse the object in the second rinse composition. Alternatively, liquid transporter (7) can include a sprayer. The sprayer can be adapted and configured to apply the second rinse composition to the object. Spraying can occur in a spray chamber portion of fifth station (43), which can be the entirety of this station. Liquid transporter (7) can include a pump and a valve adapted and configured to circulate the second rinse composition at fifth station (43), to add the second rinse composition to fifth station (43), and to remove the second rinse composition from fifth station (43). This embodiment can also include a dispenser (15) adapted and configured to add a rinse concentrate to fluid employed at fifth station (43).

In a five station embodiment, drying generally occurs at fifth station (43). Thus, fifth station (43) can include dryer (9). Various features of dryer (9) are described hereinabove. Fifth station (43) can also include the apparatus (49) that adds sterilant to and vents sterilant from system (1). Such apparatus (49) is also described hereinabove. In an embodiment, fifth station (43) includes dryer (9) and does not include a liquid transporter (7). In this embodiment, first station (3), second station (25), third station (33), and/or fourth station (37) include liquid transporter (7), sonicator (5), and other apparatus that washes, accomplishes antimicrobial treatment, and rinses (as described hereinabove).

In a five station embodiment, the five stations can occupy any of several locations with respect to one another. The five stations can share or have distinct subassemblies, such as plumbing (e.g. sprayers, pumps, etc.). For example, first station (3), second station (25), third station (33), fourth station (37), and fifth station (43) can occupy the same space, but employ different apparatus for circulating compositions. This embodiment can employ configurations analogous to the configuration described hereinabove for two, three, and four station embodiments. In another configuration, one or more of first station (3), second station (25), third station (33), fourth station (37), and/or fifth station (43) can occupy overlapping spaces. Typically fourth station (37) and fifth station (43) occupy overlapping spaces in a configuration analogous to that described hereinabove for two, three, and four station embodiments. In yet another configuration, first station (3), second station (25), third station (33), fourth station (37), and/or fifth station (43) can occupy distinct spaces. Such configurations are analogous to those described hereinabove for two, three, and four station embodiments. In embodiments in which one or more of the stations occupy the same space fourth and fifth stations (37 and 43), third, fourth and fifth stations (33, 37, and 43); second, third, fourth and fifth stations (25, 33, 37, and 43); or first second, third, fourth and fifth stations (3, 25, 33, 37, and 43), can include a (the same) spray chamber, or, for example, a (the same) tub, tank, or bath.

A five station embodiment can include an apparatus for moving the object from first station (3) to second station (25), from second station (25) to third station (33), from third station (33) to fourth station (37), from fourth station (37) to fifth station (43), among any other combination of stations, and/or through system (1). For example, system (1) can include a conveyor (31) adapted and configured to move the object between first, second, third, fourth, and/or fifth stations (3, 25, 33, 37, and 43), and/or through system (1). The five station embodiments can employ conveyor systems with capabilities and structures analogous to those described hereinabove for two, three, and four station embodiments. In a manual embodiment, an operator moves the object between first, second, third, fourth, and/or fifth stations (3, 25, 33, 37, and 43).

A five station embodiment can include one or more emitters (51) as described hereinabove for the system and one station embodiments.

In an embodiment, system (1) includes first station (3), second station (25), third station (33), fourth station (37), fifth station (43), sonicator (5), liquid transporter (7), and dryer (9). Sonicator (5) is adapted and configured to impact the object at first station (3) with ultrasonic energy. Liquid transporter (7) is adapted and configured to circulate at first station (3) and around the object a wash composition, an antimicrobial composition, a rinse composition, or a plurality of these compositions. Liquid transporter (7) is adapted and configured to circulate at second station (25) and around the object an antimicrobial composition, a rinse composition, or a plurality of these compositions. Liquid transporter (7) is adapted and configured to circulate at third station (33) and around the object an antimicrobial composition, a rinse composition, or a plurality of these compositions. Liquid transporter (7) is adapted and configured to circulate at fourth station (37) and around the object an antimicrobial composition, a rinse composition, or a plurality of these compositions. Dryer (9) is adapted and configured to dry the object at the fifth station (43), preferably in the presence of the sterilant.

In this embodiment, sonicator (5) can take on the variety of configurations described hereinabove for system (1), one, two, three, and/or four station embodiments.

In this embodiment, liquid transporter (7), or its subsystems, can circulate the wash, rinse, and antimicrobial compositions at the five stations (3, 25, 33, 37, and 43) in any of several combinations. With each combination, first station (3) includes sonicator (5). As one combination, liquid transporter (7) can be adapted and configured to circulate at first station (3) and around the object the wash composition, to circulate at second station (25) and around the object a first rinse composition, to circulate at third station (33) and around the object the antimicrobial composition, to circulate at fourth station (37) and around the object a second rinse composition.

Six Station Embodiments

System (1) can include first station (3), second station (25), third station (33), fourth station (37), fifth station (43), and a sixth station (47). In such a configuration, typically, first station (3) includes sonicator (5) and is adapted and configured to circulate at first station (3) and around the object the wash composition, the antimicrobial composition, the rinse composition, or a plurality of these compositions. In such a configuration, second station (25) is typically adapted and configured to circulate at second station (25) and around the object the antimicrobial composition, the rinse composition, or a plurality of these compositions. In such a configuration, third station (33) is typically adapted and configured to circulate at third station (33) and around the object the antimicrobial composition, the rinse composition, or a plurality of these compositions. In such a configuration, fourth station (37) can be adapted and configured to circulate at fourth station (37) and around the object the antimicrobial composition, the rinse composition, or a plurality of these compositions. In such a configuration, fifth station (43) can be adapted and configured to circulate at fifth station (43) and around the object the rinse composition, preferably a second rinse composition. In such a configuration, sixth station (47) can be adapted and configured to dry the object and/or to expose the object to gaseous sterilant. Although one or more of the stations (3, 25, 33 and 37) can each circulate one or more of the same compositions, this generally does not occur.

Sonicator (5), liquid transporter (7), holder (11) and other system (1) components can adopt configurations described hereinabove for system (1) or one, two, three, four and five station embodiments. Preferably, first station (3), sonicator (5), and liquid transporter (7) are adapted and configured to immerse the object in the wash composition and apply ultrasonic energy to the immersed object at first station (3), as described hereinabove. First station (3) can also be adapted and configured to circulate around the object a first rinse composition. Preferably, second station (25) is adapted and configured to circulate around the object the antimicrobial composition, the rinse composition, or a plurality of these compositions, as described hereinabove. Second station (25) can be adapted and configured to circulate around the object the first rinse composition. Preferably, third station (33) is adapted and configured to circulate around the object the antimicrobial composition, the rinse composition, or a plurality of these compositions, as described hereinabove. Preferably, fourth station (37) is adapted and configured to circulate around the object the antimicrobial composition, the rinse composition, or a plurality of these compositions, as described hereinabove. Fourth station (37) can be adapted and configured to circulate around the object a second rinse composition. Fifth station (43) can be adapted and configured to circulate around the object the rinse composition, preferably a second rinse composition.

In a six station embodiment, drying or exposing to gaseous sterilant generally occurs at fifth station (43). Thus, fifth station (43) can include dryer (9). Various features of dryer (9) are described hereinabove. Fifth station (43) can also include the apparatus (49) that adds sterilant to and vents sterilant from system (1). Such apparatus (49) is also described hereinabove. In an embodiment, fifth station (43) includes dryer (9) and does not include a liquid transporter (7). In an embodiment, fifth station (43) includes apparatus (49) that adds gaseous sterilant and does not include a liquid transporter (7). Typically, if fifth station (43) includes dryer (9), sixth station (47) includes apparatus (49) that adds gaseous sterilant. Typically, if fifth station (43) includes apparatus (49) that adds gaseous sterilant, sixth station (47) includes dryer (9).

In a six station embodiment, drying or exposing to gaseous sterilant generally occurs at sixth station (47). Thus, sixth station (47) can include dryer (9). Various features of dryer (9) are described hereinabove. Sixth station (47) can also include the apparatus (49) that adds sterilant to and vents sterilant from system (1). Such apparatus (49) is also described hereinabove. In an embodiment, sixth station (47) includes dryer (9) and does not include a liquid transporter (7). In an embodiment, sixth station (47) includes apparatus (49) that adds gaseous sterilant and does not include a liquid transporter (7). Typically, if sixth station (47) includes dryer (9), fifth station (43) includes apparatus (49) that adds gaseous sterilant, and vice versa. Typically, if sixth station (47) includes apparatus (49) that adds gaseous sterilant, fifth station (43) includes dryer (9).

In a six station embodiment, typically first station (3), second station (25), third station (33), and/or fourth station (37) include liquid transporter (7), sonicator (5), and other apparatus that washes, accomplishes antimicrobial treatment, and rinses (as described hereinabove).

In a six station embodiment, the six stations can occupy any of several locations with respect to one another. The six stations can share or have distinct subassemblies, such as plumbing (e.g. sprayers, pumps, etc.). For example, first station (3), second station (25), third station (33), fourth station (37), fifth station (43), and sixth station can occupy the same space, but employ different apparatus for circulating compositions or for adding and venting gaseous sterilant. This embodiment can employ configurations analogous to the configuration described hereinabove for two, three, four, and five station embodiments. In another configuration, one or more of first station (3), second station (25), third station (33), fourth station (37), fifth station (43), and/or sixth station (47) can occupy overlapping spaces. Typically fifth station (43) and sixth station (47) occupy overlapping spaces in a configuration analogous to that described hereinabove for two, three, four, and five station embodiments. In yet another configuration, first station (3), second station (25), third station (33), fourth station (37), fifth station (43), and/or sixth station (47) can occupy distinct spaces. Such configurations are analogous to those described hereinabove for two, three, four, and five station embodiments. In embodiments in which one or more of the stations occupy the same space fifth and sixth stations (43 and 47); fourth, fifth, and sixth stations (37, 43, and 47); third, fourth, fifth, and sixth stations (33, 37, 43, and 47); second, third, fourth, fifth, and sixth stations (25, 33, 37, 43, and 47); or first second, third, fourth, fifth, and sixth stations (3, 25, 33, 37, 43, and 47), can include a (the same) spray chamber, or, for example, a (the same) tub, tank, or bath. In embodiments in which one or more of the stations occupy the same space fifth and sixth stations (43 and 47) can include a (the same) gas tight chamber or chamber under negative pressure.

A six station embodiment can include an apparatus for moving the object from first station (3) to second station (25), from second station (25) to third station (33), from third station (33) to fourth station (37), from fourth station (37) to fifth station (43), from fifth station (43) to sixth station (47), among any other combination of stations, and/or through system (1). For example, system (1) can include a conveyor (31) adapted and configured to move the object between first, second, third, fourth, fifth, and/or sixth stations (3, 25, 33, 37, 43, and 47), and/or through system (1). The six station embodiments can employ conveyor systems with capabilities and structures analogous to those described hereinabove for two, three, four, and five station embodiments. In a manual embodiment, an operator moves the object between first, second, third, fourth, fifth, and/or sixth stations (3, 25, 33, 37, 43, and 47).

A six station embodiment can include one or more emitters (51) as described hereinabove for the system and one station embodiments.

In an embodiment, system (1) includes first station (3), second station (25), third station (33), fourth station (37), fifth station (43), sixth station (47), sonicator (5), liquid transporter (7), and dryer (9). Sonicator (5) is adapted and configured to impact the object at first station (3) with ultrasonic energy. Liquid transporter (7) is adapted and configured to circulate at first station (3) and around the object a wash composition, an antimicrobial composition, a rinse composition, or a plurality of these compositions. Liquid transporter (7) is adapted and configured to circulate at second station (25) and around the object an antimicrobial composition, a rinse composition, or a plurality of these compositions. Liquid transporter (7) is adapted and configured to circulate at third station (33) and around the object an antimicrobial composition, a rinse composition, or a plurality of these compositions. Liquid transporter (7) is adapted and configured to circulate at fourth station (37) and around the object an antimicrobial composition, a rinse composition, or a plurality of these compositions.

Dryer (9) is adapted and configured to dry the object at the fifth station; and apparatus (49) is adapted and configured to add to the sixth station gaseous sterilant. Alternatively, apparatus (49) is adapted and configured to add to the fifth station gaseous sterilant; and dryer (9) is adapted and configured to dry the object at the sixth station.

In this embodiment, sonicator (5) can take on the variety of configurations described hereinabove for system (1), one, two, three, four, and/or five station embodiments.

In this embodiment, liquid transporter (7), or its subsystems, can circulate the wash, rinse, and antimicrobial compositions at the four stations (3, 25, 33, and 37) in any of several combinations. With each combination, first station (3) includes sonicator (5). As one combination, liquid transporter (7) can be adapted and configured to circulate at first station (3) and around the object the wash composition, to circulate at second station (25) and around the object a first rinse composition, to circulate at third station (33) and around the object the antimicrobial composition, to circulate at fourth station (37) and around the object a second rinse composition.

Systems with more than six stations can include duplicates of the stations described above, can include analogous stations to those described above, or can include additional stations.

Embodiments Employing Other Energy Sources

Although a sonicator represents a preferred apparatus for applying energy before and during circulation of wash composition, the system (1) and methods of the present invention can employ sources of energy other than a sonicator and forms of energy other than ultrasonic energy. For example, a variety of types of energy that can be emitted form a point source can be substituted for emitting ultrasonic energy from a point source. Suitable forms of energy that can be emitted from a point source, that can contract an object, and that can aid cleaning of an object include microwave energy, continuous or pulsed light (preferably ultraviolet light) energy, and the like.

For employing such point energy sources, an emitter (51) can be substituted for sonicator (5) or second sonicator (41) in any of the embodiments described herein above. Emitter (51) can include a point source of microwave energy, a point source of light energy (e.g., ultraviolet light), preferably pulsed light energy, and the like. For example, emitter (51) can include a source of ultraviolet light, such as a mercury arc lamp or a xenon flash lamp. Such lamps can provide either continuous or burst pulsed high energy ultraviolet light. Preferably, emitter (51) includes a source of ultraviolet light in the range of about 180 nm to about 300 nm. Such an emitter can include a quartz window to shield the light source from washing composition or soil.

Method for Washing and Disinfecting

The present invention also relates to a method for washing and disinfecting an object.

This method can include one or more of contacting the object with a wash composition and energy, such as ultrasonic energy; treating the object with an antimicrobial composition; rinsing the object with a rinse composition; and/or drying the object, preferably in the presence of a sterilant. Preferably, the method of the invention includes contacting, treating, rinsing, and drying.

In an embodiment, the present method includes contacting the object with ultrasonic energy and wash composition comprising antimicrobial agent, treating the object with antimicrobial composition comprising peroxycarboxylic acid, rinsing the object with rinse composition comprising an alcohol drying agent, and drying the object in the presence of sterilant comprising chlorine dioxide. In an embodiment, the method of the invention includes contacting the object with wash composition and ultrasonic energy and providing the ultrasonic energy from point source adjacent the object, treating the object with antimicrobial composition, rinsing the object with rinse composition, drying the object, and exposing the object to gaseous sterilant. This embodiment can include providing ultrasonic energy from irrigated probe adjacent the object.

In an embodiment, the present method includes contacting the object with wash composition and ultrasonic energy, applying ultrasonic energy throughout the wash composition, treating the object with antimicrobial composition, rinsing the object with rinse composition, drying the object, and exposing the object to gaseous sterilant. In yet another embodiment of the present method, it includes applying ultrasonic energy to the object from point source adjacent the object, contacting the object with wash composition and ultrasonic energy, applying ultrasonic energy throughout the wash composition, treating the object with antimicrobial composition, rinsing the object with rinse composition, drying the object, and exposing the object to gaseous sterilant. In still another embodiment, the method of the invention includes applying ultrasonic energy to the object from point source adjacent the object, contacting the object with wash composition and ultrasonic energy, applying ultrasonic energy throughout the wash composition, rinsing the object with first rinse composition, treating the object with antimicrobial composition, rinsing the object with second rinse composition, drying the object, and exposing the object to gaseous sterilant.

The method of the invention can be carried out using a system, such as the system of the invention. In a preferred embodiment, the method includes carrying out contacting, treating, rinsing, and drying in single system. Such a single system can include a first station adapted and configured to house the object; a sonicator adapted and configured to impact the object in the first station with ultrasonic energy; a liquid transporter adapted and configured to circulate at the first station and around the object wash composition, antimicrobial composition, rinse composition, or to circulate plurality of these compositions; and a dryer adapted and configured to dry the object in the presence of sterilant.

In an embodiment, the present method includes contacting the object with wash composition and ultrasonic energy, treating the object with antimicrobial composition, rinsing the object with rinse composition, drying the object, and exposing the object to gaseous sterilant. In this embodiment, the method includes carrying out contacting, treating, rinsing, and drying in a system including a first station adapted and configured to house the object; a sonicator adapted and configured to impact the object in the first station with ultrasonic energy; a liquid transporter adapted and configured to circulate at the first station and around the object wash composition, antimicrobial composition, rinse composition, or to circulate a plurality of these compositions; and dryer adapted and configured to dry the object.

Contacting with Wash Composition and Energy

The wash composition employed in the method of the invention typically includes a cleaning composition suitable for hard surface cleaning and/or for ware washing. Preferably, the cleaning composition is milder than a cleaning composition employing caustic as a cleaning agent. Preferably, the cleaning composition does not unacceptably corrode metals, such as aluminum, steel, or stainless steel. For example, the cleaning composition can include carbonate as a cleaning agent and a metal protecting silicate to prevent or reduce corrosion.

The antimicrobial agent employed in the wash composition is effective for reducing the population of microorganisms in the wash composition and on the object, but typically cannot provide high level disinfection or sanitization during washing. Contacting with a wash composition including an antimicrobial agent provides a preliminary reduction in population of microorganisms. Suitable antimicrobial agents for the wash composition include quaternary ammonium antimicrobial agents, acid sanitizers, and other health care surface compatible antimicrobial agents, preferably one or more quaternary ammonium antimicrobial agents.

Although impacting or contacting with ultrasonic energy represents a preferred embodiment, contacting the object with energy can occur through one or more of several known mechanisms for applying energy to an object. For example, contacting can include applying microwave energy, light energy, or the like, or a combination thereof. For example, applying microwave energy can employ a point source of microwave energy that contacts the object with microwave energy before contact with the wash composition. By way of further example, applying light energy can employ a source of continuous or pulsed light, preferably ultraviolet light, that contacts the object inside or outside the system, and before or during contact with the wash composition. Applying light energy can employ a light source immersed in the wash composition and emitting light energy from a point within the wash composition, preferably a point adjacent the object, or a larger light source or multiple sources that provide illumination throughout the wash composition. Light energy can be applied before contacting the object with the wash composition and within or outside of the chamber employed for washing.

Contacting the object with ultrasonic energy occurs through one or more of several known mechanisms for applying ultrasonic energy to an object. For example, applying ultrasonic energy can employ general ultrasonication throughout the wash composition as in an ultrasonic bath, a sonicator probe immersed in the wash composition and emitting ultrasonic energy from a point within the wash composition, preferably a point adjacent the object, employing an irrigated probe that may or may not be in the wash composition but is preferably adjacent the object, and the like. Ultrasonic energy can be applied before contacting the object with the wash composition and within or outside of the chamber employed for washing. Preferably, washing also includes application of ultrasonic energy throughout the wash composition. An irrigated ultrasonic probe represents a preferred mode for applying ultrasonic energy before applying the wash composition.

Contacting an object with a wash composition typically includes circulating the wash composition around the object. As described hereinabove, circulating includes any way in which a fluid, particularly a liquid, can be moved over, in, and/or around the object. Preferably, in the present method, circulating includes sequentially circulating around the object a wash composition, an antimicrobial composition, and a rinse composition, preferably in that order. Circulating the wash composition can also include adding the wash composition to the surroundings of the object and removing the wash composition from around the object. For example, contacting can include immersing the object in the wash composition, spraying the object with a wash composition, or a combination thereof. The present method can also include forming a wash composition by adding a wash concentrate to a fluid.

Treating With Antimicrobial Composition

Treating the object with an antimicrobial agent can employ any of a variety of antimicrobial agents suitable for achieving high level disinfection or sterilization of an object such as a medical instrument or device. Suitable antimicrobial compositions include antimicrobial agents such as peroxycarboxylic acids, and the like. The method of the invention preferably employs such antimicrobial agents at a sufficient concentration and/or for a sufficient time to achieve high level disinfection or sterilization.

Treating the object with an antimicrobial agent preferably includes treating with a composition including a peroxycarboxylic acid. Certain preferred peroxycarboxylic acid compositions are described hereinbelow including in patents and patent applications incorporated by reference. For example, preferred peroxycarboxylic acids include antimicrobially effective amounts of peroxyacetic acid, peroxyoctanoic acid, peroxylactic acid, peroxynonanoic acid, peroxyheptanoic acid, and the like, or combinations thereof. Preferred peroxycarboxylic acid antimicrobial compositions also include ingredients such as a buffer capable of maintaining a use composition at pH about 5 to about 7, preferably about pH 6, such as phosphate, trisodium phosphate, or the like. Such a preferred composition preferably lacks any anticorrosive agent.

Preferably treating with a peroxycarboxylic acid antimicrobial composition occurs at about room or ambient temperature, preferably at about 20 to about 25° C. Preferably treating with a peroxycarboxylic acid antimicrobial composition occurs at a pH near or slightly below neutral pH, preferably at about pH 5 to about 7, preferably pH about 6.

Treating with an antimicrobial composition preferably achieves a higher level of antimicrobial action and can be achieved with a wash composition including an antimicrobial agent. For example, treating with an antimicrobial composition, e.g., a peroxycarboxylic acid composition, preferably achieves high level disinfection. An antimicrobial treatment can achieve high level disinfection through treatment with a suitable peroxycarboxylic acid at a sufficient concentration and/or for a sufficient time. Typically, high level disinfection can be achieved by treating with a peroxycarboxylic acid antimicrobial composition for about 10 to about 12 minutes. By way of further example, treating with an antimicrobial composition, e.g., a peroxycarboxylic acid composition, preferably achieves sterilization. An antimicrobial treatment can achieve sterilization through treatment with a suitable peroxycarboxylic acid at a sufficient concentration and/or for a sufficient time. Typically, sterilization can be achieved by treating with a peroxycarboxylic acid antimicrobial composition for more than about 12 minutes.

Treating an object with a antimicrobial composition typically includes circulating the antimicrobial composition around the object. As described hereinabove, circulating includes any way in which a fluid, particularly a liquid, can be moved over, in, and/or around the object. Circulating the antimicrobial composition can also include adding the antimicrobial composition to the surroundings of the object and removing the antimicrobial composition from around the object. For example, treating can include immersing the object in the antimicrobial composition, spraying the object with an antimicrobial composition, or a combination thereof. The present method can also include forming a antimicrobial composition by adding a antimicrobial concentrate to a fluid.

Treating can include applying energy to the object during or after circulating the antimicrobial composition. Treating can include, for example, applying light or microwave energy to the object, preferably in the presence of the antimicrobial composition. For example, treating can include applying light, preferably ultraviolet light, energy throughout the antimicrobial composition. By way of further example, treating can include applying light, preferably ultraviolet light, energy or microwave energy after circulating the antimicrobial composition.

Rinsing

Rinsing with a rinse composition can achieve one or more of several goals. Rinsing removes excess wash composition and/or antimicrobial composition from the object. Preferably, rinsing leaves the object free of any unwanted residue of the wash composition and/or the antimicrobial composition after rinsing and drying. Preferably, the rinse composition includes water, an alcohol drying agent, a lubricant, a rinse agent, or a combination thereof. Rinsing preferably includes a first rinsing and/or a second rinsing, preferably both. First rinsing preferably occurs after contacting with wash composition and before treating with antimicrobial composition. Second rinsing preferably occurs after treating with antimicrobial composition and before drying. Different rinse compositions can be employed for first rinsing and second rinsing. For example, first rinsing can employ a composition including water, a rinse agent, or combination thereof. First rinsing preferably prepares the wash object for antimicrobial treatment. Second rinsing preferably employs a composition including an alcohol drying agent, a lubricant, a rinse agent, or a combination thereof. Second rinsing preferably prepares the treated object for drying.

Rinsing an object with a rinse composition typically includes circulating the rinse composition around the object. As described hereinabove, circulating includes any way in which a fluid, particularly a liquid, can be moved over, in, and/or around the object. Circulating the rinse composition can also include adding the rinse composition to the surroundings of the object and removing the rinse composition from around the object. For example, rinsing can include immersing the object in the rinse composition, spraying the object with an rinse composition, or a combination thereof. The present method can also include forming a rinse composition by adding a rinse concentrate to a fluid.

Drying

Drying can achieve one or more of several goals. First, drying can remove any remaining liquid from antimicrobial treatment and/or rinsing from the object. Second, drying in the presence of a sterilant can both remove liquid from and remove any remaining living or functional organisms from the object. Preferably, drying includes exposing the object to a heated dry fluid, such as heated air. Preferably, drying in the presence of a sterilant includes both drying and exposing the object to a sterilant, preferably a gaseous sterilant. Alternatively, exposing the object to a sterilant can occur before or after drying.

Exposing to Sterilant

The method can employ any sterilant suitable for disinfecting or sterilizing medical instruments or devices. Preferred gaseous sterilants include chlorine dioxide, ethylene oxide, ozone, plasma, and the like, or a combination thereof. Exposing the object to the gaseous sterilant typically includes releasing the gaseous sterilant through a pressure release valve from a pressurized vessel and into the surroundings of the object. The gaseous disinfectant contacts the object. Drying and/or exposing can also include evacuating the gaseous sterilant from around the object and, optionally, from the system.

Exposing to sterilant can, in certain embodiments, be supplemented by or replaced by applying energy to the object before, after, or during operation of the dryer. For example, after circulating (second) rinse composition, the method can include applying energy to the object. For example, during drying, the method can include applying energy to the object. For example, after drying, the method can include applying energy to the object.

Translocating

The method can include moving the object from one location to another, or translocating the object, between one or more of contacting, treating, rinsing, and drying. For example, the method can include one or more of translocating the object between contacting and treating, between treating and rinsing, between rinsing and drying. Translocating can also occur between any other processes included in the method. For example, when the method includes contacting the object with ultrasonic energy before contacting with the wash composition, the object can be translocated between the site of contacting with ultrasonic energy of the site of contacting with the wash composition. When the method includes, a first rinsing between contacting and treating and/or a second rinsing between treating and drying, translocating can occur at one or more of between washing and the first rinsing, between the first rinsing and contacting, between treating and the second rinsing, and between the second rinsing and drying. Translocating the object can employ any of several apparatus suitable for moving an object or a rack containing an object from one location to another, such as a conveyor.

Compositions Employed in the Method and System

Wash Composition

The system and method of the present invention can employ wash compositions typically employed for cleaning objects such as medical or dental instruments or devices. As described hereinabove, the wash composition typically contains an antimicrobial agent, such as a quaternary ammonium antimicrobial agent. Suitable wash compositions include solid wash compositions and liquid wash compositions, acid wash compositions and alkaline wash compositions. Preferred wash compositions include compositions of solid carbonate cleaning compositions.

Solid Carbonate Cleaning Compositions

The present systems and methods can employ solid carbonate cleaning compositions, which are typically solids based on a matrix of carbonate and bicarbonate, but including additional ingredients. Suitable solid carbonate cleaning compositions are described, for example in U.S. Pat. Nos. 6,177,392 and 6,156,715 and in U.S. patent applications Ser. Nos. 08/989,824, entitled BINDING AGENT FOR SOLID BLOCK FUNCTIONAL MATERIAL, and ______, filed Feb. 1, 2001 and entitled STABLE SOLID ENZYME COMPOSITIONS AND METHODS EMPLOYING THEM, the disclosures of which are incorporated herein by reference.

Wash Composition Antimicrobial Agents

The present wash compositions and methods employing them can include any of a variety of antimicrobial agents. Such antimicrobial agents include quaternary ammonium antimicrobial agents, acid sanitizers, and other health care surface compatible antimicrobial agents.

Quaternary ammonium antimicrobial agents are useful in the present invention, due to their commercial availability, easy incorporation into formulas and high sanitizing efficacy. These sanitizing agents are also preferred because of their compatibility to high water temperatures to the presence of high organic loads, stability and broad spectrum antimicrobial efficacy in variable high and low pH wash systems, inherent chemical deodorizing, and their non-staining, non-bleaching, non-corrosive nature. Illustrative quaternary ammonium salts include distearyl dimethyl ammonium chloride, stearyl dimethyl benzyl ammonium chloride, coconut alkyl dimethyl benzyl ammonium chloride, dicoconut alkyl dimethyl ammonium bromide, cetyl pyridinium iodide, and cetyl pyridinium iodide, and cetyl trimethyl ammonium bromide, and the like.

Suitable fatty acids for the wash composition include a health care surface compatible aliphatic or aromatic fatty acid, either saturated or unsaturated, preferably, saturated, and having from about 6 to about 20 carbon atoms and, preferably, from about 8 to about 12 carbon atoms, as well as mixtures thereof The fatty acid may be linear, branched or cyclic and may contain substituent atoms such as hydroxyl groups or ether linkages as long as the substituents do not affect antimicrobial activity. Representative of the fatty acids contemplated for use herein include caproic acid, caprylic acid, capric acid, lauric acid, and octanoic acid as well as mixtures thereof.

Other medical instrument compatible antimicrobial agents include aldehyde antimicrobial agents; carboxylic acid antimicrobial agents; peracid and peroxygen antimicrobial agents; ozone; organic halogen, inorganic halogen, neutral oxide of a halogen (e.g. chlorine dioxide), and halogen releasing antimicrobial agents, such as iodine, iodine complexes, interhalogens, chlorine sodium trichloroisocyanurate, iodinepoly(vinylpyrolidinonen) complexes, and 2-bromo-2-nitropropane-1,3-diol; phenolic antimicrobial agents (e.g., pentachlorophenol and orthophenylphenol); surface-active antimicrobial agents, such as acid-anionic, amphoteric and cationic surfactants; nitrogen containing antimicrobial agents and polymers, such as alkylamines alkanol amines, nitro derivatives, and analides; metal derivatives; organosulfur and sulfur-nitrogen compounds; and the like; and mixtures thereof. These various antimicrobial agents are known to those of skill in the art and can be employed in the compositions and methods of the present invention.

Alkyl phosphate esters possess some antimicrobial activity in their own right under the conditions of the present invention. This antimicrobial activity also tends to add to the overall antimicrobial activity of the present compositions even though the phosphate esters may be added for other reasons.

Antimicrobial Composition

The system and method of the present invention can employ antimicrobial compositions typically employed for cleaning, disinfecting, and/or sterilizing objects such as medical or dental instruments or devices. Suitable antimicrobial compositions include solid antimicrobial compositions and liquid antimicrobial compositions. Many of the antimicrobial agents described above for use in the wash composition can also be used at the same or higher levels, with other added ingredients, and for longer exposure times to yield disinfection or sterilization of an object, such as a medical or dental instrument or device.

Preferred antimicrobial compositions include peroxycarboxylic acids.

Peroxycarboxylic Acid Antimicrobial Composition

A variety of liquid peroxycarboxylic acid antimicrobial compositions are known to those of skill in the art and can be employed in the methods of the present invention. For example, suitable compositions are disclosed in U.S. Pat. Nos. 6,010,729, issued Jan. 4, 2000 to Gutzmann et al.; 5,718,910, issued Feb. 17, 1998 to Oakes et al.; 5,674,538, issued May 24, 1994 to Lokkesmoe et al.; 5,489,434, issued Feb. 6, 1996 to Oakes et al.; 5,437,868, issued Aug. 1, 1995 to Oakes et al.; 5,409,713, issued Apr. 25, 1995 to Lokkesmoe et al.; 5,314,687, issued May 24, 1994 to Oakes et al.; and 5,200,189, issued Apr. 6, 1993 to Oakes et al.; which are incorporated herein by reference for disclosure of these compositions and methods for applying them. One preferred composition is disclosed in U.S. patent application Ser. No. 09/447,328, filed Nov. 22, 1999 and entitled NON-CORROSIVE STERILANT COMPOSITION, which is incorporated herein by reference.

A preferred antimicrobial composition of the present invention includes acetic acid, octanoic acid, peroxyacetic acid, and peroxyoctanoic acid. Such a composition can also include a chelating agent. A preferred composition preferably includes a combination of peroxyacetic acid and peroxyoctanoic acid effective for killing one or more pathogenic bacteria, such as Salmonella typhimurium, Escherichia coli, Staphylococcus aureus, Salmonella choleraesuis, Pseudomonas aeruginosa, yeast, mold, viruses, mycobacteria, fungi, and the like. The compositions and methods of the present invention have activity against a wide variety of microorganisms such as Gram positive (for example, Staphylococcus aureus) and Gram negative (for example, Pseudomonas aeruginosa) bacteria, yeast, molds, bacterial spores, viruses, mycobacteria, fungi, etc. The compositions and methods of the present invention, as described above, have activity against a wide variety of human pathogens. The compositions and methods can kill a wide variety of microbes on the surface of an object or in water used for washing or processing the object.

Rinse Compositions

The system and method of the present invention can employ rinse compositions typically employed for rinsing objects such as medical or dental instruments or devices. Suitable rinse compositions include solid rinse compositions and liquid rinse compositions, neutral rinse compositions and neutralizing rinse compositions.

The rinse composition typically contains a wetting or sheeting agent combined with other optional ingredients. The rinse composition includes a water soluble or dispersible low foaming organic material capable of reducing the surface tension of the rinse water to promote sheeting action and to prevent spotting or streaking caused by beaded water after rinsing is complete in washing or treating processes. Such sheeting agents are typically organic surfactant like materials having a characteristic cloud point.

The cloud point of the surfactant rinse or sheeting agent is defined as the temperature at which a 1 wt. % aqueous solution of the surfactant turns cloudy when warmed. Preferred surfactants employed in the present rinse compositions a cloud point greater than the temperature of available hot service water. Accordingly, the lowest useful cloud point measured for the surfactants of the invention is approximately 40° C. The cloud point can also be 60° C. or higher, 70° C. or higher, 80° C. or higher, etc., depending on the use locus hot water temperature and the temperature and type of rinse cycle.

Preferred sheeting agents, typically include a polyether compound prepared from ethylene oxide, propylene oxide, or a mixture in a homopolymer or block or heteric copolymer structure. Such polyether compounds are known as polyalkylene oxide polymers, polyoxyalkylene polymers or polyalkylene glycol polymers. Such sheeting agents require a region of relative hydrophobicity and a region of relative hydrophilicity to provide surfactant properties to the molecule. Such sheeting agents have a molecular weight in the range of about 500 to 15,000. Certain types of (PO)(EO) polymeric rinse aids have been found to be useful containing at least one block of poly(PO) and at least one block of poly(EO) in the polymer molecule. Additional blocks of poly(EO), poly PO or random polymerized regions can be formed in the molecule.

Rinse compositions employed in the present invention can include one or more nonionic surfactants, such as one or more EO PO copolymers, urea, and one or more silicones, such as one or more silicone dimethyl polysiloxane compounds. Aspects of the such rinse compositions are described in U.S. Pat. No. 4,624,713, to Morganson, et al., issued Nov. 25, 1986, and entitled SOLID RINSE AIDS AND METHODS OF WAREWASHING UTILIZING SOLID RINSE AIDS; the disclosure of which is incorporated herein by reference. Rinse compositions employed in the present invention can include a nonionic block copolymer composition, defoamer composition, and a water soluble casting agent. Aspects of such rinse compositions are described in U.S. Pat. No. 5,589,099, to Baum, issued Dec. 31, 1996, and entitled LOW FOAMING RINSE AGENTS COMPRISING ETHYLENE OXIDE/PROPYLENE OXIDE BLOCK COPOLYMER; the disclosure of which is incorporated herein by reference.

Disinfectant or Sterilant

The system and method of the present invention can employ gaseous disinfectants or sterilants typically employed for disinfecting and/or sterilizing objects such as medical or dental instruments or devices. Certain of the antimicrobial agents described above for use in the wash composition can be employed as a gaseous disinfectant or sterilant. Suitable gaseous disinfectants or sterilants include chlorine dioxide, ethylene oxide, ozone, plasma, and the like, or combinations thereof. Preferred gaseous disinfectants or sterilants include chlorine dioxide, ethylene oxide, ozone, and the like. The present system and method can also employ a disinfectant or sterilant in the drying cycle that will dissipate during the drying cycle and leave no unacceptable residue on the object.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration. The phrase “adapted and configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

We claim:

1. A system for washing, disinfecting, or sterilizing an object, the system comprising:
first station adapted and configured to house the object;
sonicator adapted and configured to impact the object in the first station with ultrasonic energy;
liquid transporter adapted and configured to circulate at the first station and around the object wash composition, antimicrobial composition, rinse composition, or to circulate a plurality of these compositions; and
dryer adapted and configured to dry the object.

2. The system of claim 1, wherein the sonicator is adapted and configured to provide ultrasonic energy from a point at the first station.

3. The system of claim 2, further comprising holder at the first station, the holder being adapted and configured for supporting the object, and
wherein the sonicator comprises ultrasonic probe at the first station and adjacent position taken by the object in the holder.

4. The system of claim 2, wherein the sonicator comprises irrigated probe.

5. The system of claim 1, wherein the sonicator is adapted and configured to provide ultrasonic energy throughout fluid at the first station.

6. The system of claim 5, further comprising second sonicator adapted and configured to provide ultrasonic energy from probe outside the first station.

7. The system of claim 6, wherein the second sonicator comprises irrigated probe.

8. The system of claim 6, further comprising holder adapted and configured to support the object adjacent the second sonicator probe.

9. The system of claim 1, wherein the sonicator is adapted and configured to dislodge soil from the object and the liquid transporter is adapted and configured to remove the soil.

10. The system of claim 1, wherein the liquid transporter comprises pump and valve adapted and configured to circulate fluid at the first station and around the object, to add fluid to the first station, and to remove fluid from the first station.

11. The system of claim 1, wherein the first station, the sonicator, and the liquid transporter are adapted and configured to immerse the object in the wash composition and apply ultrasonic energy to the immersed object.

12. The system of claim 11, wherein the sonicator is adapted and configured to provide ultrasonic energy from point within the first station.

13. The system of claim 11, wherein the sonicator comprises irrigated probe.

14. The system of claim 11, wherein the sonicator applies ultrasonic energy throughout the wash composition.

15. The system of claim 11, wherein the first station comprises tub, tank, or bath.

16. The system of claim 1, wherein the first station and the liquid transporter are adapted and configured to immerse the object in the antimicrobial composition, the rinse composition, or a plurality of these compositions.

17. The system of claim 16, wherein the first station comprises tub, tank, or bath.

18. The system of claim 1, wherein the liquid transporter comprises sprayer adapted and configured to apply to the object the antimicrobial composition, the rinse composition, or a plurality of these compositions.

19. The system of claim 18, wherein the first station comprises spray chamber.

20. The system of claim 1, wherein the liquid transporter is adapted and configured to sequentially circulate the wash composition, the antimicrobial composition, and the rinse composition.

21. The system of claim 1, wherein the liquid transporter is adapted and configured to circulate the wash composition, the antimicrobial composition, first rinse composition, second rinse composition, or to circulate a plurality of these compositions.

22. The system of claim 21, wherein the liquid transporter is adapted and configured to sequentially circulate, in this order, the wash composition, the first rinse composition, the antimicrobial composition, and the second rinse composition.

23. The system of claim 1, further comprising dispenser adapted and configured to add wash concentrate, antimicrobial concentrate, rinse concentrate, or to add a plurality of these concentrates.

24. The system of claim 1, wherein the dryer is adapted and configured to dry the object at the first station.

25. The system of claim 1, wherein the dryer comprises blower adapted and configured to circulate heated gas at the first station.

26. The system of claim 1, further comprising injector adapted and configured to add sterilant at the first station.

27. The system of claim 26, wherein the injector comprises pressure release valve adapted and configured to release at the first station gaseous sterilant from pressurized vessel.

28. The system of claim 26, further comprising vent adapted and configured to evacuate sterilant gas from the first station.

29. The system of claim 1, further comprising conveyor adapted and configured to transport the object through the system.

30. The system of claim 1, further comprising emitter adapted and configured to impact the object with microwave or light energy.

31. The system of claim 1, further comprising second station, wherein:
the first station comprises the sonicator and is adapted and configured to circulate at the first station and around the object the wash composition, the antimicrobial composition, the rinse composition, or a plurality of these compositions; and
the second station is adapted and configured to circulate at the second station and around the object the antimicrobial composition, the rinse composition, or a plurality of these compositions; the second station comprises the dryer; or a combination thereof.

32. The system of claim 31, wherein the liquid transport system comprises sprayer at the second station and adapted and configured to apply to the object the antimicrobial composition, the rinse composition, or a plurality of these compositions.

33. The system of claim 32, wherein the second station comprises spray chamber.

34. The system of claim 31, wherein the second station and the liquid transporter are adapted and configured to immerse the object in the antimicrobial composition, the rinse composition, or a plurality of these compositions.

35. The system of claim 34, wherein the second station comprises tub, tank, or bath.

36. The system of claim 31, wherein the second station is adapted and configured to circulate around the object the rinse composition.

37. The system of claim 36, wherein the first station is adapted and configured to circulate around the object the wash composition, the antimicrobial composition, and first rinse composition; and the second station is adapted and configured to circulate around the object second rinse composition.

38. The system of claim 31, wherein the second station is adapted and configured to circulate around the object the antimicrobial composition and the rinse composition.

39. The system of claim 38, wherein the first station is adapted and configured to circulate around the object the wash composition and first rinse composition; and the second station is adapted and configured to circulate around the object the antimicrobial composition and second rinse composition.

40. The system of claim 31, wherein the second station comprises the dryer.

41. The system of claim 31, further comprising injector adapted and configured to add sterilant at the second station.

42. The system of claim 31, further comprising third station, wherein:
the first station comprises the sonicator and is adapted and configured to circulate at the first station and around the object the wash composition, the antimicrobial composition, or a plurality of these compositions;
the second station is adapted and configured to circulate at the second station and around the object the antimicrobial composition, the rinse composition, or a plurality of these compositions; and
the third station is adapted and configured to circulate at the third station and around the object the antimicrobial composition, the rinse composition, or a plurality of these compositions; the third station comprises the dryer; or a combination thereof.

43. The system of claim 42, wherein the third station comprises the dryer.

44. The system of claim 43, further comprising injector adapted and configured to add sterilant at the third station.

45. The system of claim 42, wherein the third station is adapted and configured to circulate around the object the rinse composition.

46. The system of claim 45, wherein the liquid transport system comprises sprayer at the third station and adapted and configured to apply the rinse composition to the object.

47. The system of claim 46, wherein the third station comprises spray chamber.

48. The system of claim 45, wherein the third station and the liquid transporter are adapted and configured to immerse the object in the rinse composition.

49. The system of claim 48, wherein the third station comprises tub, tank, or bath.

50. The system of claim 42, wherein:
the first station comprises the sonicator and is adapted and configured to circulate around the object the wash composition, first rinse composition and the antimicrobial composition; and
the second station is adapted and configured to circulate around the object second rinse composition.

51. The system of claim 42, wherein:
the first station comprises the sonicator and is adapted and configured to circulate around the object the wash composition and first rinse composition; and
the second station is adapted and configured to circulate around the object the antimicrobial composition and second rinse composition.

52. The system of claim 42, wherein:
the first station comprises the sonicator and is adapted and configured to circulate around the object the wash composition; and
the second station is adapted and configured to circulate around the object first rinse composition, the antimicrobial composition, and second rinse composition.

53. The system of claim 42, wherein:
the first station comprises the sonicator and is adapted and configured to circulate around the object the wash composition and first rinse composition;
the second station is adapted and configured to circulate around the object the antimicrobial composition; and
the third station is adapted and configured to circulate around the object second rinse composition.

54. The system of claim 42, wherein:
the first station comprises the sonicator and is adapted and configured to circulate around the object the wash composition;
the second station is adapted and configured to circulate around the object first rinse composition and the antimicrobial composition; and
the third station is adapted and configured to circulate around the object second rinse composition.

55. The system of claim 42, further comprising fourth station, wherein:
the first station comprises the sonicator and is adapted and configured to circulate at the first station and around the object the wash composition, the antimicrobial composition, or a plurality of these compositions;
the second station is adapted and configured to circulate at the second station and around the object the antimicrobial composition, the rinse composition, or a plurality of these compositions;
the third station is adapted and configured to circulate at the third station and around the object the antimicrobial composition, the rinse composition, or a plurality of these compositions; and
the fourth station is adapted and configured to circulate at the fourth station and around the object the antimicrobial composition, the rinse composition, or a plurality of these compositions; the fourth station comprises the dryer; or a combination thereof.

56. The system of claim 55, wherein the fourth station comprises the dryer.

57. The system of claim 56, further comprising injector adapted and configured to add sterilant at the fourth station.

58. The system of claim 55, wherein the fourth station is adapted and configured to circulate around the object the rinse composition.

59. The system of claim 58, wherein the liquid transport system comprises sprayer in the fourth station and adapted and configured to apply the rinse composition to the object.

60. The system of claim 59, wherein the fourth station comprises spray chamber.

61. The system of claim 58, wherein the fourth station and the liquid transporter are adapted and configured to immerse the object in the rinse composition.

62. The system of claim 61, wherein the fourth station comprises tub, tank, or bath.

63. The system of claim 55, wherein:
the first station comprises the sonicator and is adapted and configured to circulate around the object the wash composition;
the second station is adapted and configured to circulate around the object the antimicrobial composition and first rinse composition; and
the third station is adapted and configured to circulate around the object second rinse composition.

64. The system of claim 55, wherein:
the first station comprises the sonicator and is adapted and configured to circulate around the object the wash composition and first rinse composition;
the second station is adapted and configured to circulate around the object the antimicrobial composition;
the third station is adapted and configured to circulate around the object second rinse composition.

65. The system of claim 55, wherein:
the first station comprises the sonicator and is adapted and configured to circulate around the object the wash composition;
the second station is adapted and configured to circulate around the object first rinse composition; and
the third station is adapted and configured to circulate around the object the antimicrobial composition and second rinse composition.

66. The system of claim 55, wherein:
the first station comprises the sonicator and is adapted and configured to circulate around the object the wash composition;
the second station is adapted and configured to circulate around the object first rinse composition;
the third station is adapted and configured to circulate around the object the antimicrobial composition; and
the fourth station is adapted and configured to circulate around the object second rinse composition and the fourth station comprises the dryer.

67. The system of claim 55, further comprising fifth station, wherein:
the first station comprises the sonicator and is adapted and configured to circulate at the first station and around the object the wash composition, the antimicrobial composition, or a plurality of these compositions;
the second station is adapted and configured to circulate at the second station and around the object the antimicrobial composition, the rinse composition, or a plurality of these compositions;
the third station is adapted and configured to circulate at the third station and around the object the antimicrobial composition, the rinse composition, or a plurality of these compositions;
the fourth station is adapted and configured to circulate at the fourth station and around the object the antimicrobial composition, the rinse composition, or a plurality of these compositions;
the fifth station is adapted and configured to circulate at the fifth station and around the object the rinse composition, the fifth station comprises the dryer, or a combination thereof.

68. The system of claim 67, wherein the fifth station comprises the dryer.

69. The system of claim 68, further comprising injector adapted and configured to add sterilant at the fifth station.

70. The system of claim 67, wherein the fifth station is adapted and configured to circulate around the object the rinse composition.

71. The system of claim 70, wherein the liquid transport system comprises sprayer in the fifth station and adapted and configured to apply the rinse composition to the object.

72. The system of claim 71, wherein the fifth station comprises spray chamber.

73. The system of claim 70, wherein the fifth station and the liquid transporter are adapted and configured to immerse the object in the rinse composition.

74. The system of claim 73, wherein the fifth station comprises tub, tank, or bath.

75. The system of claim 67, wherein:
the first station comprises the sonicator and is adapted and configured to circulate the wash composition;
the second station is adapted and configured to circulate first rinse composition;
the third station is adapted and configured to circulate the antimicrobial composition; and
the fourth station is adapted and configured to circulate second rinse composition.

76. The system of claim 67, further comprising sixth station, wherein:
the first station comprises the sonicator and is adapted and configured to circulate at the first station and around the object the wash composition, the antimicrobial composition, or a plurality of these compositions;
the second station is adapted and configured to circulate at the second station and around the object the antimicrobial composition, the rinse composition, or a plurality of these compositions;
the third station is adapted and configured to circulate at the third station and around the object the antimicrobial composition, the rinse composition, or a plurality of these compositions;
the fourth station is adapted and configured to circulate at the fourth station and around the object the antimicrobial composition, the rinse composition, or a plurality of these compositions;
the fifth station is adapted and configured to circulate at the fifth station and around the object the rinse composition, the fifth station comprises the dryer, the fifth station comprises the apparatus to add gaseous sterilant, or a combination thereof; and
the sixth station comprises the dryer, the sixth station comprises the apparatus to add gaseous sterilant, or a combination thereof.

77. A system for washing, disinfecting, or sterilizing an object, the system comprising:
sonicator adapted and configured to impact the object with ultrasonic energy;
liquid transporter adapted and configured to circulate around the object wash composition, antimicrobial composition, and rinse composition; and
dryer adapted and configured to dry the object in the presence of sterilant.

78. A system for washing, disinfecting, or sterilizing object, the system comprising:
first station and second station;
sonicator adapted and configured to impact the object in the first station with ultrasonic energy;
liquid transporter adapted and configured to circulate at the first station and around the object wash composition, antimicrobial composition, rinse composition, or plurality of these compositions; and
dryer adapted and configured to dry the object in the second station in the presence of sterilant.

79. A system for washing, disinfecting, or sterilizing an object, the system comprising:
first station, second station, and third station;
sonicator adapted and configured to impact the object in the first station with ultrasonic energy;
liquid transporter adapted and configured to circulate at the first station and around the object wash composition, antimicrobial composition, rinse composition, or a plurality of these compositions; and to circulate at the second station and around the object the antimicrobial composition, the rinse composition, or a plurality of these compositions; and
dryer adapted and configured to dry the object in the third station in the presence of sterilant.

80. A system for washing, disinfecting, or sterilizing an object, the system comprising:
first station, second station, third station, and fourth station;
sonicator adapted and configured to impact the object in the first station with ultrasonic energy;
liquid transporter adapted and configured to circulate at the first station and around the object wash composition, antimicrobial composition, rinse composition, or a plurality of these compositions; to circulate at the second station and around the object the antimicrobial composition, the rinse composition, or a plurality of these compositions; and to circulate at the third station and around the object the antimicrobial composition, the rinse composition, or a plurality of these compositions; and
dryer adapted and configured to dry the object in the fourth station in the presence of sterilant.

81. A system for washing, disinfecting, or sterilizing an object, the system comprising:
first station, second station, third station, fourth station, and fifth station;
sonicator adapted and configured to impact the object in the first station with ultrasonic energy;
liquid transporter adapted and configured to circulate at the first station and around the object wash composition, antimicrobial composition, rinse composition, or plurality of these compositions; to circulate at the second station and around the object the antimicrobial composition, the rinse composition, or plurality of these compositions; to circulate at the third station and around the object the antimicrobial composition, the rinse composition, or plurality of these compositions; and to circulate at the fourth station and around the object the antimicrobial composition, the rinse composition, or a plurality of these compositions; and
dryer adapted and configured to dry the object at the fifth station in the presence of sterilant.

82. A system for washing, disinfecting, or sterilizing an object, the system comprising:
first station, second station, third station, fourth station, fifth station, and sixth station;
sonicator adapted and configured to impact the object in the first station with ultrasonic energy;
liquid transporter adapted and configured to circulate at the first station and around the object wash composition, antimicrobial composition, rinse composition, or plurality of these compositions; to circulate at the second station and around the object the antimicrobial composition, the rinse composition, or plurality of these compositions; to circulate at the third station and around the object the antimicrobial composition, the rinse composition, or plurality of these compositions; and to circulate at the fourth station and around the object the antimicrobial composition, the rinse composition, or a plurality of these compositions;
dryer adapted and configured to dry the object at the fifth station; and
apparatus adapted and configured to add to the sixth station gaseous sterilant.

83. A system for washing, disinfecting, or sterilizing an object, the system comprising:
first station, second station, third station, fourth station, fifth station, and sixth station;
sonicator adapted and configured to impact the object in the first station with ultrasonic energy;
liquid transporter adapted and configured to circulate at the first station and around the object wash composition, antimicrobial composition, rinse composition, or plurality of these compositions; to circulate at the second station and around the object the antimicrobial composition, the rinse composition, or plurality of these compositions; to circulate at the third station and around the object the antimicrobial composition, the rinse composition, or plurality of these compositions; and to circulate at the fourth station and around the object the antimicrobial composition, the rinse composition, or a plurality of these compositions;
apparatus adapted and configured to add to the fifth station gaseous sterilant; and
dryer adapted and configured to dry the object at the sixth station.

84. A system for washing, disinfecting, or sterilizing an object, the system comprising:
emitter adapted and configured to impact the object with energy;
liquid transporter adapted and configured to circulate around the object wash composition, antimicrobial composition, and rinse composition; and
dryer adapted and configured to dry the object in the presence of sterilant.

85. The system of claim 84, wherein the emitter comprises source of ultraviolet light adapted and configured to impact the object with ultraviolet light.

86. The system of claim 84, wherein the emitter comprises source of microwave energy adapted and configured to impact the object with microwave energy.

87. The system of claim 84, wherein the emitter and the liquid transporter are adapted and configured to impact the object with energy during or after circulating antimicrobial agent.

88. The system of claim 84, wherein the emitter and the dryer are adapted and configured to impact the object with energy before, during, or after operation of the dryer.

89. A method for washing, disinfecting, or sterilizing an object, the method comprising:
contacting the object with wash composition and ultrasonic energy;
treating the object with antimicrobial composition;
rinsing the object with rinse composition;
drying the object; and
exposing the object to gaseous sterilant.

90. The method of claim 89, wherein the wash composition comprises antimicrobial agent.

91. The method of claim 89, wherein the wash composition comprises solid carbonate cleaning composition.

92. The method of claim 89, wherein contacting the object with ultrasonic energy comprises providing ultrasonic energy from point source adjacent the object.

93. The method of claim 92, wherein contacting the object with ultrasonic energy comprises providing ultrasonic energy from irrigated probe adjacent the object.

94. The method of claim 89, wherein contacting the object with ultrasonic energy comprises applying ultrasonic energy throughout the wash composition.

95. The method of claim 89, wherein contacting the object with ultrasonic energy comprises providing ultrasonic energy from point source adjacent the object before contacting the object with the wash composition and applying ultrasonic energy throughout the wash composition.

96. The method of claim 95, wherein providing ultrasonic energy from point source adjacent the object comprises providing ultrasonic energy from irrigated probe adjacent the object.

97. The method of claim 89, wherein treating comprises treating with antimicrobial composition comprising peroxycarboxylic acid.

98. The method of claim 97, wherein the peroxycarboxylic acid comprises peroxyacetic acid, peroxyoctanoic acid, peroxyheptanoic acid, peroxynonanoic acid, or a combination thereof.

99. The method of claim 97, wherein treating comprises treating the object for sufficient time to achieve high level disinfection.

100. The method of claim 97, wherein treating comprises treating the object for sufficient time to achieve sterilization.

101. The method of claim 89, wherein treating comprises immersing the object in the antimicrobial composition, spraying the object with the antimicrobial composition, or a combination thereof.

102. The method of claim 89, wherein treating comprises impacting the object with microwave or light energy.

103. The method of claim 89, wherein the rinse composition comprises an alcohol drying agent, lubricant, rinse agent, or a combination thereof.

104. The method of claim 89, wherein rinsing comprises first rinsing after contacting and before treating, second rinsing after treating and before drying, or a combination thereof.

105. The method of claim 89, wherein rinsing comprises immersing the object in the rinse composition, spraying the object with the rinse composition, or a combination thereof.

106. The method of claim 89, wherein drying comprises circulating heated gas around the object.

107. The method of claim 89, wherein drying comprises exposing the object to gaseous sterilant.

108. The method of claim 89, wherein the gaseous sterilant comprises chlorine dioxide, ethylene oxide, or a combination thereof.

109. The method of claim 108, wherein drying comprises releasing the gaseous sterilant through pressure release valve from pressurized vessel.

110. The method of claim 108, wherein drying further comprises evacuating the gaseous sterilant from around the object.

111. The method of claim 89, wherein drying comprises impacting the object with microwave or light energy.

112. The method of claim 89, further comprising translocating the object between contacting and treating, between treating and rinsing, between rinsing and drying, or a combination thereof.

113. The method of claim 89, comprising carrying out contacting, treating, rinsing, and drying in system comprising:
first station adapted and configured to house the object;
sonicator adapted and configured to impact the object in the first station with ultrasonic energy;
liquid transporter adapted and configured to circulate at the first station and around the object wash composition, antimicrobial composition, rinse composition, or to circulate plurality of these compositions; and
dryer adapted and configured to dry the object in the presence of sterilant.

114. A method for washing, disinfecting, or sterilizing an object, the method comprising:
contacting the object with wash composition and ultrasonic energy and providing the ultrasonic energy from point source adjacent the object;
treating the object with antimicrobial composition;
rinsing the object with rinse composition;
drying the object; and
exposing the object to gaseous sterilant.

115. A method for washing, disinfecting, or sterilizing an object, the method comprising:
contacting the object with wash composition and ultrasonic energy;
applying ultrasonic energy throughout the wash composition;
treating the object with antimicrobial composition;
rinsing the object with rinse composition;
drying the object; and
exposing the object to gaseous sterilant.

116. A method for washing, disinfecting, or sterilizing an object, the method comprising:
applying ultrasonic energy to the object from point source adjacent the object;
contacting the object with wash composition and ultrasonic energy;
applying ultrasonic energy throughout the wash composition;
treating the object with antimicrobial composition;
rinsing the object with rinse composition;
drying the object; and
exposing the object to gaseous sterilant.

117. A method for washing, disinfecting, or sterilizing an object, the method comprising:
applying ultrasonic energy to the object from point source adjacent the object;
contacting the object with wash composition and ultrasonic energy;
applying ultrasonic energy throughout the wash composition;
rinsing the object with first rinse composition;
treating the object with antimicrobial composition;
rinsing the object with second rinse composition;
drying the object; and
exposing the object to gaseous sterilant.

118. A method for washing, disinfecting, or sterilizing an object, the method comprising:
contacting the object with wash composition and emitted energy;
treating the object with antimicrobial composition;
rinsing the object with rinse composition;
drying the object; and
exposing the object to gaseous sterilant.
